(19) United States
(12) Patent Application Publication
(10) Pub. No.: US 2011/0311980 A1
(43) Pub. Date: Dec. 22, 2011

(54) Nucleic Acid Amplification and Sequencing on a Droplet Actuator

(75) Inventors: Michael G. Pollack, Durham, NC (US); Allen E. Eckhardt, Durham, NC (US); Vijay Srinivasan, Durham, NC (US); Prasanna Thwar, Los Altos, CA (US)

(73) Assignee: ADVANCED LIQUID LOGIC, INC., Research Triangle Park, NC (US)

(21) Appl. No.: 13/139,467

(22) PCT Filed: Dec. 15, 2009
(86) PCT No.: PCT/US09/68040

§ 371 Date: Aug. 17, 2011

Related U.S. Application Data
(60) Provisional application No. 61/122,622, filed on Dec. 15, 2008.
(60) Provisional application No. 61/144,765, filed on Jan. 15, 2009.
(60) Provisional application No. 61/141,387, filed on Dec. 30, 2008.
(60) Provisional application No. 61/140,377, filed on Dec. 23, 2008.

(51) Int. Cl. C12Q 1/68; C12M 1/34

(52) U.S. Cl. 435/6.12; 435/287.2; 435/6.1

(57) ABSTRACT

The invention provides a droplet actuator device, as well as systems, methods and devices making use of the droplet actuator device. The droplet actuator device may include a substrate having electrodes arranged for conducting one or more droplet operations. The droplet actuator device may include a substrate having a reactor path with a wash region associated with a magnet for immobilizing mobilizing beads during bead washing operations. The droplet actuator device may include nucleotide base reservoirs and dedicated nucleotide base electrode paths arranged for transporting nucleotide base droplets from nucleotide base reservoirs to the reactor path. The droplet actuator device may include one or more wash buffer reservoirs associated with electrode paths arranged for transporting wash buffer droplets from wash buffer reservoirs to the reactor path. The droplet actuator device may include one or more sample reservoirs and sample paths arranged for transporting sample droplets from the one or more sample reservoirs to the reactor path. The droplet actuator device may include one or more enzyme reservoirs and dedicated enzyme electrode paths arranged for transporting enzyme droplets from the one or more enzyme reservoirs to a detection electrode.

### 2 RELATED APPLICATIONS

In addition to the patent applications cited herein, each of which is incorporated herein by reference, this patent application is related to and claims priority to U.S. Provisional Patent Application Nos. 61/122,622, filed on Dec. 15, 2008, entitled “Miniaturized Nucleic Acid Sequencer for Identification of Microbial Pathogens;” 61/140,377, filed on Dec. 23, 2008, entitled “Devices and Methods for Droplet-Based Nucleic Acid Amplification and Sequencing;” 61/141,387, filed on Dec. 30, 2008, entitled “Devices and Methods for Droplet-Based Nucleic Acid Amplification and Sequencing;” and 61/144,765, filed on Jan. 15, 2009, entitled “Devices and Methods for Droplet-Based Nucleic Acid Amplification and Sequencing;” the entire disclosures of which are incorporated herein by reference.

### 1 GRANT INFORMATION

This invention was made with government support under HG003706 awarded by the National Institutes of Health. The United States Government has certain rights in the invention.

### 3 FIELD OF THE INVENTION

The invention relates generally to devices and methods for amplifying and/or sequencing nucleic acid using droplet operations on a droplet actuator.

### 4 BACKGROUND OF THE INVENTION

Droplet actuators are used to conduct a wide variety of droplet operations. A droplet actuator typically includes one or more substrates configured to form a surface or gap for conducting droplet operations. The one or more substrates include electrodes for conducting droplet operations. The gap between the substrates is typically filled or coated with a filler fluid that is immiscible with the liquid that is to be subjected to droplet operations. Droplet operations are controlled by electrodes associated with the one or more substrates. There is a need for techniques that make use of droplet actuators for amplification and sequencing of nucleic acids.

### 5 BRIEF DESCRIPTION OF THE INVENTION

The invention provides a droplet actuator device, as well as systems, methods and devices making use of the droplet actuator device. The droplet actuator device may include a substrate having electrodes arranged for conducting one or more droplet operations. The droplet actuator device may include a substrate having a reactor path with a wash region associated with a magnet for immobilizing beads during bead washing operations. The droplet actuator device may include nucleotide base reservoirs and dedicated nucleotide base electrode paths arranged for transporting nucleotide base droplets from nucleotide base reservoirs to the reactor path. The droplet actuator device may include one or more wash buffer reservoirs associated with electrode paths arranged for transporting wash buffer droplets from wash buffer reservoirs to the reactor path. The droplet actuator device may include one or more sample reservoirs and sample paths arranged for transporting sample droplets from the one or more sample reservoirs to the reactor path. The droplet actuator device may include one or more enzyme reservoirs and dedicated enzyme electrode paths arranged for transporting enzyme droplets from the one or more enzyme reservoirs to a detection electrode. A dedicated path is a path which does not intersect with another path. In various embodiments, at least a portion of the electrode paths for each nucleotide is dedicated in the sense that it does not intersect with any of the other nucleotide reagent paths. In various embodiments, at least a portion of the electrode paths for each sample is dedicated in the sense that it does not intersect with any of other sample paths.

The droplet actuator may include one or more nucleic acid sample droplets in the one or more sample reservoirs.

The droplet actuator may include a nucleic acid sample droplet having one or more beads with a primed nucleic acid bound thereto. In some cases, the nucleic acid sample droplet includes more than about 100 magnetically-responsive beads. In some cases, the nucleic acid sample droplet includes less than about 100 magnetically-responsive beads. In some cases, the nucleic acid sample droplet includes less than 10 magnetically-responsive beads. In some cases, the nucleic acid sample droplet includes less than 5 magnetically-responsive beads. In some cases, the nucleic acid sample droplet includes a single magnetically-responsive bead.

In certain embodiments, the nucleic acid sample droplet has a volume that is less than about 500 pL. In certain embodiments, the nucleic acid sample droplet has a volume that is less than about 50 pL. In certain embodiments, the nucleic acid sample droplet has a volume that is less than about 5 pL. In certain embodiments, the nucleic acid sample droplet has a volume that is approximately 1 pL.

The droplet actuator may have one or more enzyme droplets in the one or more enzyme reservoirs. The one or more enzyme droplets may include one or more enzymes selected from the group consisting of DNA polymerases, ATP sulfurylases, and luciferases. The one or more enzyme droplets may include one or more PPi detection enzymes. The PPi detection enzymes may include a sulfurylase enzyme and a luciferase enzyme. The one or more enzyme droplets may include nucleotide base incorporation enzymes.

The droplet actuator may have nucleotide base droplets in the one or more nucleotide base reservoirs.

In certain embodiments, the electrodes may have a diameter in the range of about 1 μm to about 500 μm. In certain embodiments, the electrodes may have a diameter in the range of about 1 μm to about 250 μm. In certain embodiments, the electrodes may have a diameter in the range of about 1 μm to about 100 μm. In certain embodiments, the electrodes may have a diameter less than about 100 μm. In some cases, the pyrosequencing reaction is conducted using droplets that may have a volume which may be less than about 1 mL. In other cases, the droplets may have a volume which may be less than about 500 μL. In other cases, the droplets may have a volume which may be less than about 50 μL.

The invention also provides a system having a processor electronically coupled to the electrodes of the droplet actuator and programmed to execute one or more sequencing protocols using droplet operations affected by the electrodes. The system may be programmed to execute one or more pyrosequencing protocols using droplet operations affected by the electrodes.

The invention provides a droplet actuator having a PCB substrate having electrodes configured for conducting droplet operations. The PCB substrate may be subjected to one or more remedial measures effecting reduced background noise caused by PPi contamination relative to a corresponding PCB substrate lacking the remedial measures. The remedial measures may be selected to reduce background noise caused by PPi contamination to an extent sufficient to eliminate undue interference with a pyrosequencing reaction conducted on the droplet actuator. The remedial measures may reduce PPi contamination sufficiently to eliminate undue interference of background PPi with detection of PPi generated by a pyrosequencing reaction. In some embodiments, the remedial measures may include selecting a PCB material manufactured without a pyrophosphate treatment or with a reduced treatment sufficient to eliminate undue interference of background PPi from the PCB with detection of PPi generated by the pyrosequencing reaction. In some embodiments, the remedial measures may include subjecting the PCB to procedures in the droplet actuator manufacturing process to reduce the introduction of PPi contamination. In some embodiments, the remedial measures may include washing or otherwise treating the PCB to reduce PPi contamination. In some embodiments, the remedial measures may include washing or otherwise treating the PCB to reduce PPi contamination using a solution which chemically modifies, inactivates, absorbs and/or removes some or all of the PPi. In some embodiments, the remedial measures may include washing the PCB in an acid bath to reduce PPi contamination. In some embodiments, the remedial measures may include treating the PCB with an enzyme to reduce PPi contamination. The enzyme may, for example, include a pyrophosphatase. In some embodiments, the remedial measures may include coating the PCB or a region of the PCB with a substance that blocks PPi release. The coating that is used to block PPi release may include a hydrophobic coating. The coating that is used to block PPi release may include a surface coating selected from the group consisting of: TEFLON® coatings, CYTOP® coatings, silane coatings, and silicone coatings. The surface coating may have a thickness sufficient to eliminate undue interference of background PPi from the PCB with detection of PPi generated by the pyrosequencing reaction.

The invention provides a method of identifying a base at a target position in a sample nucleic acid. The method may include providing a droplet actuator having a droplet actuator substrate having electrodes arranged for conducting one or more droplet operations a sample single stranded nucleic acid immobilized on a nucleic acid substrate. The method may include combining on the droplet actuator (1) a droplet having an amplified DNA template hybridized to a sequencing primer and coupled to one or more beads with (2) a droplet having a nucleotide, APS and luciferin to yield a bead and nucleotide-containing droplet. The method may include combining on the droplet actuator a droplet having DNA polymerase, ATP sulfurylase and luciferase with the bead and nucleotide-containing droplet to yield a reaction droplet. The method may include detecting on the droplet actuator a signal from the reaction droplet. The method may include transporting the reaction droplet into the presence of a detector prior to step detecting on the droplet actuator a signal from the reaction droplet.

Detecting a signal from the reaction droplet may include detecting incorporation of a nucleotide as a luminescent signal proportional to the number of adjacent bases incorporated into the strand being synthesized. Detecting a signal from the reaction droplet may include detecting a non-incorporated nucleotide as a background signal. The method may include washing the beads following detecting on the droplet actuator a signal from the reaction droplet.

The method may include repeating the chain extension sequence with different nucleotides using a cyclic nucleotide dispensing strategy. The sequence may be repeated with different nucleotides using an ordered nucleotide dispensing strategy based on a reference template. The sequence may be repeated with different nucleotides, wherein each subsequent nucleotide may be selected based on the statistical probability that such nucleotide may be likely to be successfully incorporated. In certain embodiments, the pyrosequencing methods may be repeated with different nucleotides using an ordered nucleotide dispensing strategy based on a reference template.

A common buffer formulation may be used as a wash buffer for washing the beads following step detection. The method may include supplying supplemental polymerase to the bead and nucleotide-containing droplet to replace polymerase that may be dislodged during washing steps. For example, the droplet having a nucleotide, APS and luciferin may also include supplemental polymerase to replace polymerase that may be dislodged during washing steps.

The invention provides a method of conducting a nucleotide base incorporation reaction. The method may include providing a sample droplet on a droplet actuator in the presence of a magnetic field. The sample droplet may include one or more magnetically-responsive beads. The one or more magnetically-responsive beads may include a DNA-primer complex bound thereto. The DNA-primer complex may include a target DNA bound to a primer. The method may include washing the beads on the droplet actuator to yield a washed-bead droplet having washed beads having the DNA-primer complex. The method may include combining on the droplet actuator the washed-bead droplet with one or more droplets having a nucleotide base and one or more substrates to yield a nucleotide base droplet. The method may include combining the nucleotide base droplet with one or more enzyme droplets to yield a detection droplet. The enzyme droplet may include enzymes sufficient to incorporate a nucleotide base into the DNA-primer complex and catalyze the generation of a signal using the substrates. Incorporation of the nucleotide base produces signal proportional to the number of adjacent bases incorporated. Non-incorporation of the nucleotide base produces a signal which may be less than the signal produced by the incorporation of a single base.

The one or more enzyme droplets may include one or more enzymes selected from the group consisting of DNA polymerases, ATP sulfurylases, and luciferases. The one or more enzyme droplets may include one or more PPi detection enzymes. The one or more enzyme droplets may include enzyme preparations selected to produce no PPi background or PPi background that does not cause undue interference in the detection of PPi from the nucleotide base incorporation reaction. The PPi detection enzymes may include a sulfurylase enzyme and a luciferase enzyme. The one or more enzyme droplets may include nucleotide base incorporation enzymes.

The one or more droplets having a nucleotide base and one or more substrates may include APS in a concentration selected to yield from about 1 to about 20 μM APS in the detection droplet. The one or more droplets having a nucleotide base and one or more substrates may include APS in a concentration selected to yield from about 5 to about 15 μM APS in the detection droplet. The one or more droplets having a nucleotide base and one or more substrates may include APS in a concentration selected to yield from about 8 to about 12 μM APS in the detection droplet. The one or more enzyme droplets may include luciferin in a concentration selected to yield from about 25 to about 75 ng/μL luciferin in the detection droplet. The one or more enzyme droplets may include luciferin in a concentration selected to yield from about 35 to about 65 ng/μL luciferin in the detection droplet. The one or more enzyme droplets may include luciferin in a concentration selected to yield from about 45 to about 55 ng/μL luciferin in the detection droplet.

Combining the nucleotide base droplet with one or more enzyme droplets to yield a detection droplet may include transporting the enzyme droplet into proximity with a detector during or prior to combining the washed-bead droplet with one or more droplets comprising a nucleotide base and one or more substrates to yield a nucleotide base droplet.

One or more of the steps of any of the methods of the invention may be mediated at least in part by electrodes, e.g., electrowetting-mediated or dielectrophoresis mediated. One or more of the steps of the methods of the invention may be accomplished using droplet operations with droplets positioned in a gap between two droplet actuator substrates. The gap may include a filler fluid. The filler fluid may, for example, be selected from the group consisting of: silicone oils; fluorosilicone oils; hydrocarbons; aliphatic and aromatic alkanes; halogenated oils; mixtures of any of the foregoing oils in the same class; and mixtures of any of the foregoing oils in different classes.

Washing the beads to yield a washed-bead droplet comprising washed beads comprising the DNA-primer complex may include conducting droplet operations on the droplet actuator to merge a wash droplet with the sample droplet having the beads to yield a merged droplet; substantially immobilizing or otherwise restraining the beads in the merged droplet; and conducting droplet operations to separate a droplet from the merged droplet thereby carrying away unbound substances from the beads. Washing may be repeated until a predetermined concentration of unbound substances may be achieved. One or more of the wash droplets may include apyrase. In other embodiments, the wash droplets may specifically exclude apyrase.

Combining the washed-bead droplet with one or more droplets comprising a nucleotide base and one or more substrates to yield a nucleotide base droplet may include combining on the droplet actuator the washed-bead droplet with one droplet having a nucleotide base and one or more substrates to yield the nucleotide base droplet. Combining the nucleotide base droplet with one or more enzyme droplets to yield a detection droplet may include combining the nucleotide base droplet with a single enzyme droplet to yield the detection droplet.

In some cases, signal detection is accomplished at a detection zone on the droplet actuator. The detection zone may, in some embodiments, be washed before and/or after detecting the signal. Washing the detection zone may include transporting one or more wash droplets onto and off of the detection zone. The wash droplet may, in some embodiments, include pyrophosphatase. The wash droplet may include pyrophosphatase beads. In some embodiments, the methods of the invention may include detecting signal from a detection droplet for a period which may be less than about 60 seconds. In other embodiments, the methods may include detecting signal from a detection droplet for a period which may be less than about 30 seconds. In other embodiments, the methods may include detecting signal from a detection droplet for a period which may be less than about 10 seconds. In some embodiments, detecting the signal may include flash detection. In other embodiments, detecting the signal may include glow detection.

In certain embodiments, the droplet operations steps of the method are conducted using unit-sized electrodes having a diameter in the range of about 1 μm to about 500 μm. In other embodiments, the droplet operations steps of the method are conducted using unit-sized electrodes having a diameter in the range of about 1 μm to about 250 μm. In other embodiments, the droplet operations steps of the method are conducted using unit-sized electrodes having a diameter in the range of about 1 μm to about 100 μm. In other embodiments, the droplet operations steps of the method are conducted using unit-sized electrodes having a diameter of about 100 μm.

### 6 DEFINITIONS

As used herein, the following terms have the meanings indicated.

“Activate” with reference to one or more electrodes means effecting a change in the electrical state of the one or more electrodes which, in the presence of a droplet, results in a droplet operation.

“Bead,” with respect to beads on a droplet actuator, means any bead or particle that is capable of interacting with a droplet on or in proximity with a droplet actuator. Beads may be any of a wide variety of shapes, such as spherical, generally spherical, egg shaped, disc shaped, cubical and other three dimensional shapes. The bead may, for example, be capable of being transported in a droplet on a droplet actuator or otherwise configured with respect to a droplet actuator in a manner which permits a droplet on the droplet actuator to be brought into contact with the bead, on the droplet actuator and/or off the droplet actuator. Beads may be manufactured using a wide variety of materials, including for example, resins, and polymers. The beads may be any suitable size, including for example, microbeads, microparticles, nanobeads and nanoparticles. In some cases, beads are magnetically responsive; in other cases beads are not significantly magnetically responsive. For magnetically responsive beads, the magnetically responsive material may constitute substantially all of a bead or one component only of a bead. The remainder of the bead may include, among other things, polymeric material, coatings, and moieties which permit attachment of an assay reagent. Examples of suitable magnetically responsive beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® particles, available from Invitrogen Corp., Carlsbad, Calif.), fluorescent microparticles and nanoparticles, coated magnetic microparticles and nanoparticles, ferromagnetic microparticles and nanoparticles, coated ferromagnetic microparticles and nanoparticles, and those described in U.S. Patent Publication No. 20050260686, entitled, “Multiplex flow assays preferably with magnetic particles as solid phase,” published on Nov. 24, 2005, the entire disclosure of which is incorporated herein by reference for its teaching concerning magnetically responsive materials and beads. Beads may be pre-coupled with a biomolecule (ligand). The ligand may, for example, be an antibody, protein or antigen, DNA/RNA probe or any other molecule with an affinity for the desired target. Examples of droplet actuator techniques for immobilizing magnetically responsive beads and/or non-magnetically responsive beads and/or conducting droplet operations protocols using beads are described in U.S. patent application Ser. No. 11/639,566, entitled “Droplet-Based Particle Sorting,” filed on Dec. 15, 2006; U.S. patent application Ser. No. 61/039,183, entitled “Multiplexing Bead Detection in a Single Droplet,” filed on Mar. 25, 2008; U.S. Patent Application No. 61/047,789, entitled “Droplet Actuator Devices and Droplet Operations Using Beads,” filed on Apr. 25, 2008; U.S. Patent Application No. 61/086,183, entitled “Droplet Actuator Devices and Methods for Manipulating Beads,” filed on Aug. 5, 2008; International Patent Application No. PCT/US2008/053545, entitled “Droplet Actuator Devices and Methods Employing Magnetic Beads,” filed on Feb. 11, 2008; International Patent Application No. PCT/US2008/058018, entitled “Bead-based Multiplexed Analytical Methods and Instrumentation,” filed on Mar. 24, 2008; International Patent Application No. PCT/US2008/058047, “Bead Sorting on a Droplet Actuator,” filed on Mar. 23, 2008; and International Patent Application No. PCT/US2006/047486, entitled “Droplet-based Biochemistry,” filed on Dec. 11, 2006; the entire disclosures of which are incorporated herein by reference. Bead characteristics may be employed in the multiplexing aspects of the invention. Examples of beads having characteristics suitable for multiplexing, as reservoir as methods of detecting and analyzing signals emitted from such beads, may be found in U.S. Patent Publication No. 20080305481, entitled “Systems and Methods for Multiplex Analysis of PCR in Real Time,” published on Dec. 11, 2008; U.S. Patent Publication No. 20080151240, “Methods and Systems for Dynamic Range Expansion,” published on Jun. 26, 2008; U.S. Patent Publication No. 20070207513, entitled “Methods, Products, and Kits for Identifying an Analyte in a Sample,” published on Sep. 6, 2007; U.S. Patent Publication No. 20070064990, entitled “Methods and Systems for Image Data Processing,” published on Mar. 22, 2007; U.S. Patent Publication No. 20060159962, entitled “Magnetic Microspheres for use in Fluorescence-based Applications,” published on Jul. 20, 2006; U.S. Patent Publication No. 20050277197, entitled “Microparticles with Multiple Fluorescent Signals and Methods of Using Same,” published on Dec. 15, 2005; and U.S. Patent Publication No. 20050118574, entitled “Multiplexed Analysis of Clinical Specimens Apparatus and Method,” published on Jun. 2, 2005. When working with magnetically responsive beads, it is helpful to adjust the strength of the magnetic field (e.g., by magnet selection and/or adjusting the distance from the magnet to the beads) to facilitate aggregation of the beads, without pulling them completely out of the droplet or causing irreversible bead clumping.

“Droplet” means a volume of liquid on a droplet actuator that is at least partially bounded by filler fluid. For example, a droplet may be completely surrounded by filler fluid or may be bounded by filler fluid and one or more surfaces of the droplet actuator. Droplets may, for example, be aqueous or non-aqueous or may be mixtures or emulsions including aqueous and non-aqueous components. Droplets may take a wide variety of shapes; nonlimiting examples include generally disc shaped, slug shaped, truncated sphere, ellipsoid, spherical, partially compressed sphere, hemispherical, ovoid, cylindrical, and various shapes formed during droplet operations, such as merging or splitting or formed as a result of contact of such shapes with one or more surfaces of a droplet actuator. For examples of droplet fluids that may be subjected to droplet operations using the approach of the invention, see International Patent Application No. PCT/US 06/47486, entitled, “Droplet-Based Biochemistry,” filed on Dec. 11, 2006. In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. Moreover, a droplet may include a reagent, such as water, deionized water, saline solutions, acidic solutions, basic solutions, detergent solutions and/or buffers. Other examples of droplet contents include reagents, such as a reagent for a biochemical protocol, such as a nucleic acid amplification protocol, an affinity-based assay protocol, an enzymatic assay protocol, a sequencing protocol, and/or a protocol for analyses of biological fluids.

“Droplet Actuator” means a device for manipulating droplets. For examples of droplet actuators, see U.S. Pat. No. 6,911,132, entitled “Apparatus for Manipulating Droplets by Electrowetting-Based Techniques,” issued on Jun. 28, 2005 to Pamula et al.; U.S. patent application Ser. No. 11/343,284, entitled “Apparatuses and Methods for Manipulating Droplets on a Printed Circuit Board,” filed on filed on Jan. 30, 2006; U.S. Pat. No. 6,773,566, entitled “Electrostatic Actuators for Microfluidics and Methods for Using Same,” issued on Aug. 10, 2004 and U.S. Pat. No. 6,565,727, entitled “Actuators for Microfluidics Without Moving Parts,” issued on Jan. 24, 2000, both to Shenderov et al.; Pollack et al., International Patent Application No. PCT/US2006/047486, entitled “Droplet-Based Biochemistry,” filed on Dec. 11, 2006; and Roux et al., U.S. Patent Pub. No. 20050179746, entitled “Device for Controlling the Displacement of a Drop Between two or Several Solid Substrates,” published on Aug. 18, 2005; the disclosures of which are incorporated herein by reference. Certain droplet actuators will include a substrate, droplet operations electrodes associated with the substrate, one or more dielectric and/or hydrophobic layers atop the substrate and/or electrodes forming a droplet operations surface, and optionally, a top substrate separated from the droplet operations surface by a gap. One or more reference electrodes may be provided on the top and/or bottom substrates and/or in the gap. In various embodiments, the manipulation of droplets by a droplet actuator may be electrode mediated, e.g., electrowetting mediated or dielectrophoresis mediated or Coulombic force mediated. Examples of other methods of controlling fluid flow that may be used in the droplet actuators of the invention include devices that induce hydrodynamic fluidic pressure, such as those that operate on the basis of mechanical principles (e.g. external syringe pumps, pneumatic membrane pumps, vibrating membrane pumps, vacuum devices, centrifugal forces, piezoelectric/ultrasonic pumps and acoustic forces); electrical or magnetic principles (e.g. electroosmotic flow, electrokinetic pumps, ferrofluidic plugs, electrohydrodynamic pumps, attraction or repulsion using magnetic forces and magnetohydrodynamic pumps); thermodynamic principles (e.g. gas bubble generation/phase-change-induced volume expansion); other kinds of surface-wetting principles (e.g. electrowetting, and optoelectrowetting, as reservoir as chemically, thermally, structurally and radioactively induced surface-tension gradients); gravity; surface tension (e.g., capillary action); electrostatic forces (e.g., electroosmotic flow); centrifugal flow (substrate disposed on a compact disc and rotated); magnetic forces (e.g., oscillating ions causes flow); magnetohydrodynamic forces; and vacuum or pressure differential. In certain embodiments, combinations of two or more of the foregoing techniques may be employed in droplet actuators of the invention.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles.

“Filler fluid” means a fluid associated with a droplet operations substrate of a droplet actuator, which fluid is sufficiently immiscible with a droplet phase to render the droplet phase subject to electrode-mediated droplet operations. The filler fluid may, for example, be a low-viscosity oil, such as silicone oil. Other examples of filler fluids are provided in International Patent Application No. PCT/US2006/047486, entitled, “Droplet-Based Biochemistry,” filed on Dec. 11, 2006; International Patent Application No. PCT/US2008/072604, entitled “Use of additives for enhancing droplet actuation,” filed on Aug. 8, 2008; and U.S. Patent Publication No. 20080283414, entitled “Electrowetting Devices,” filed on May 17, 2007; the entire disclosures of which are incorporated herein by reference. The filler fluid may fill the entire gap of the droplet actuator or may coat one or more surfaces of the droplet actuator. Filler fluid may be conductive or non-conductive.

“Immobilize” with respect to magnetically responsive beads, means that the beads are substantially restrained in position in a droplet or in filler fluid on a droplet actuator. For example, in one embodiment, immobilized beads are sufficiently restrained in position to permit execution of a splitting operation on a droplet, yielding one droplet with substantially all of the beads and one droplet substantially lacking in the beads.

“Magnetically responsive” means responsive to a magnetic field. “Magnetically responsive beads” include or are composed of magnetically responsive materials. Examples of magnetically responsive materials include paramagnetic materials, ferromagnetic materials, ferrimagnetic materials, and metamagnetic materials. Examples of suitable paramagnetic materials include iron, nickel, and cobalt, as reservoir as metal oxides, such as Fe₃O₄, BaFe₁₂O₁₉, CoO, NiO, Mn₂O₃, Cr₂O₃, and CoMnP.

“Transporting into the magnetic field of a magnet,” “transporting towards a magnet,” and the like, as used herein to refer to droplets and/or magnetically responsive beads within droplets, is intended to refer to transporting into a region of a magnetic field capable of substantially attracting magnetically responsive beads in the droplet. Similarly, “transporting away from a magnet or magnetic field,” “transporting out of the magnetic field of a magnet,” and the like, as used herein to refer to droplets and/or magnetically responsive beads within droplets, is intended to refer to transporting away from a region of a magnetic field capable of substantially attracting magnetically responsive beads in the droplet, whether or not the droplet or magnetically responsive beads is completely removed from the magnetic field. It will be appreciated that in any of such cases described herein, the droplet may be transported towards or away from the desired region of the magnetic field, and/or the desired region of the magnetic field may be moved towards or away from the droplet. Reference to an electrode, a droplet, or magnetically responsive beads being “within” or “in” a magnetic field, or the like, is intended to describe a situation in which the electrode is situated in a manner which permits the electrode to transport a droplet into and/or away from a desired region of a magnetic field, or the droplet or magnetically responsive beads is/are situated in a desired region of the magnetic field, in each case where the magnetic field in the desired region is capable of substantially attracting any magnetically responsive beads in the droplet. Similarly, reference to an electrode, a droplet, or magnetically responsive beads being “outside of” or “away from” a magnetic field, and the like, is intended to describe a situation in which the electrode is situated in a manner which permits the electrode to transport a droplet away from a certain region of a magnetic field, or the droplet or magnetically responsive beads is/are situated in away from a certain region of the magnetic field, in each case where the magnetic field in such region is capable of substantially attracting any magnetically responsive beads in the droplet.

“Washing” with respect to washing a bead means reducing the amount and/or concentration of one or more substances in contact with the bead or exposed to the bead from a droplet in contact with the bead. The reduction in the amount and/or concentration of the substance may be partial, substantially complete, or even complete. The substance may be any of a wide variety of substances; examples include target substances for further analysis, and unwanted substances, such as components of a sample, contaminants, and/or excess reagent. In some embodiments, a washing operation begins with a starting droplet in contact with a magnetically responsive bead, where the droplet includes an initial amount and initial concentration of a substance. The washing operation may proceed using a variety of droplet operations. The washing operation may yield a droplet including the magnetically responsive bead, where the droplet has a total amount and/or concentration of the substance which is less than the initial amount and/or concentration of the substance. Examples of suitable washing techniques are described in Pamula et al., U.S. Pat. No. 7,439,014, entitled “Droplet-Based Surface Modification and Washing,” granted on Oct. 21, 2008, the entire disclosure of which is incorporated herein by reference.

The terms “top,” “bottom,” “over,” “under,” and “on” are used throughout the description with reference to the relative positions of components of the droplet actuator, such as relative positions of top and bottom substrates of the droplet actuator. It will be appreciated that the droplet actuator is functional regardless of its orientation in space.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

### 7 BRIEF DESCRIPTION OF THE DRAWINGS

FIG. 1 illustrates a top view of an example of an electrode arrangement of an embodiment of a droplet actuator of the invention;

FIG. 2 illustrates a process of performing a pyrosequencing reaction protocol;

FIG. 3 shows a pyrogram of on-actuator pyrosequencing results of 17-bp sequenced on a 211-bp long C. albicans DNA template using the cyclic nucleotide dispensing;

FIGS. 4 and 5 show plots of ATP calibration with the diaphragm removed from the optical path;

FIG. 6 shows a plot of the dependence of chemiluminescent signal intensity on the concentration of substrates used to convert PPi to light;

FIG. 7 shows a plot of fluorescence of the FAM-labeled primer/DNA attached to beads monitored with washes;

FIG. 8 illustrates top and side views of a high-capacity reservoir design incorporating a reservoir assembly including a reservoir positioned above the on-droplet actuator reservoir to provide a constant liquid feed;

FIG. 9 illustrates a top view of an electrode arrangement of a droplet actuator organized into a unit cell that includes a single reaction zone;

FIG. 10 illustrates a top view of an electrode arrangement of a droplet actuator organized into a unit cell that includes four separate reaction zones;

FIGS. 11A and 11B illustrate top views of the alignment of the electrode arrangement of FIG. 10 with a magnetic plate;

FIG. 12 illustrates a side view of a portion of a capillary device and an alternative method for performing a pyrosequencing reaction;

FIGS. 13A and 13B are illustrations of a droplet actuator cartridge; and

FIGS. 14A and 14B show plots of real-time PCR curves obtained for a C. albicans model system.

### 8 DETAILED DESCRIPTION OF THE INVENTION

The invention provides droplet actuator devices, systems and techniques for amplifying and/or sequencing nucleic acids. Systems of the invention may include a droplet actuator and components necessary for the operation of the droplet actuator along with software for executing amplification and/or sequencing protocols. Examples of system configurations and components suitable for use with the invention are described in Smith et al., U.S. Patent Publication No. 20080281471, entitled “Droplet Actuator Analyzer with Cartridge” published on Nov. 13, 2008; as well as Paik et al., U.S. Patent Publication No. 20080006535, entitled “System for Controlling a Droplet Actuator,” published on Jan. 10, 2008; the entire disclosures of which are incorporated herein by reference. Other examples are provided herein.

8.1 Sequencing

The invention provides droplet actuator devices, systems and techniques for sequencing nucleic acids. Examples of droplet actuator configurations, reagents and protocol steps suitable for use with the present invention are described in Pollack et al., International Patent Publication No. WO/2007/120240, entitled “Droplet-Based Pyrosequencing,” published on Oct. 25, 2007, the entire disclosure of which is incorporated herein by reference.

8.1.1 Droplet Actuator Configurations

Among other things, the droplet actuator may include reservoirs for holding and dispensing reagents and/or sample; as well as one or more sequencing modules. The sequencing module(s) may, for example, include a washing zone, a sequencing zone and a detection zone.

The droplet actuator architecture may include external and/or internal reagent and/or sample reservoirs. Internal reservoirs are at least partially located within the droplet operations gap of a droplet actuator. External reservoirs are generally external to the droplet operations gap, and are associated with a fluid passage extending from the external reservoir into the droplet operations gap. Reservoirs may be associated with droplet dispensing electrode configurations. Droplet dispensing electrode configurations may be proximate to one or more transport pathways of droplet operations electrodes configured for transporting droplets across a droplet operations surface, e.g., into a sequencing module. In one specific, non-limiting embodiment, the reservoirs were as follows: primary wash reservoir (15.25 mm×22.52 mm); post-detection wash reservoir (8 mm×22.52 mm); reagent reservoir (7.75 mm×7.75 mm); PPi detection enzyme reservoir (7.75 mm×7.75 mm) Loading volumes were as follows: reagent reservoir, 50 μL; PPi detection enzyme reservoir, 75 μL; primary wash reservoir, 600 μL; and post-detection wash reservoir, 175 μL. These dimensions and volumes are only examples; other volumes and dimensions will be readily apparent in view of the instant disclosure.

Appropriate external reservoir loading volumes may be calculated using a variety of techniques. For a small volume reservoir, load a minimum volume of liquid close to the estimated dead volume. Dispense droplets from the reservoir, and when it ceases to dispense, the remaining volume left in the reservoir is the first determination of the dead volume. Refill the reservoir with a volume that is larger than the volume of dispensed droplets and repeat dispensing until it ceases. Every time it ceases to dispense, determine the dead volume. Continue until the reservoir stops dispensing because of overfill. The final loading volume is the maximum loading volume. Another protocol used for determining the dead volumes of the reservoirs independent of each other involves loading different reservoirs with different loading volumes and dispensing droplets continuously till they cease to dispense. The number of droplets vs. loading volume can be plotted to determine the dead volume, or the dead volumes of each of the reservoirs can be averaged to give the average dead volume over a range of loading volume. For a larger reservoir, a suitable protocol involves loading a volume of liquid less than the estimated dead volume of the reservoir, attempting to dispense, and filling the reservoir with smaller increments of volume until it begins to dispense. This loading volume then provides the first determination of the dead volume. Loading further past this volume and dispensing droplets until dispensing stops, gives the next determination of the dead volume. Continuing to refill until dispensing stops because of overfill (just like the previous technique), gives the maximum loading volume.

The droplet actuator architecture may include a sequencing module. The sequencing module may include electrodes, bead retention means, and/or other structures suitable for executing a sequencing protocol.

The sequencing module may include a washing zone. The washing zone may include a means for immobilizing or restraining beads during washing operations. Means for immobilizing or restraining beads may, for example, include physical obstacles and/or magnetic means for immobilizing beads during washing operations. Examples of bead immobilizing or restraining techniques suitable for use in the present invention are included in Sista et al., International Patent Publication No. WO/2008/098236, entitled “Droplet Actuator Devices and Methods Employing Magnetic Beads,” published on Aug. 14, 2008; Thwar et al., International Patent Application No. PCT/US08/74151, entitled “Bead Manipulations on a Droplet Actuator,” filed on Aug. 25, 2008; and Pamula et al., U.S. Pat. No. 7,439,014, entitled “Droplet-Based Surface Modification and Washing,” granted on Oct. 21, 2008. Where a magnet is used, the magnet may, for example, include a permanent magnet and/or an electromagnet. It is also envisioned that DNA may also be attached to a solid surface of the chip.

The sequencing module may include a reaction zone. The reaction zone is preferably located proximally to the washing zone. When a permanent magnet is used, the reaction zone is preferably, though not necessarily, located at a sufficient distance from the magnet to avoid interference in the sequencing reaction by the magnet.

The sequencing module may include a detection zone. In some embodiments, the droplet actuator and the droplet actuator instrument are configured such that when the droplet actuator is coupled to the droplet actuator instrument, the detection zone is aligned with a detector, i.e., the detector is positioned or may readily be positioned at a locus which permits detection of a signal from a droplet in the detection zone. In various embodiments, the detector may be provided on the droplet actuator, on a droplet actuator cartridge, on an instrument controlling the droplet actuator, or on a separate instrument altogether. As an example, the detector may include a photoluminescent detector. Other examples of suitable detectors include those described in Pollack et al., International Patent Publication No. WO/2007/120240, entitled “Droplet-Based Pyrosequencing,” published on Oct. 25, 2007, the entire disclosure of which is incorporated herein by reference.

Using the electrode transport pathways, reagents and buffer droplets may be added or removed from the sequencing module according to a user-defined program. In some cases, the droplet actuator may be configured and/or used to conduct a variety of pyrosequencing protocols. In other cases, the droplet actuator may be configured and/or used to conduct a specific pyrosequencing protocol.

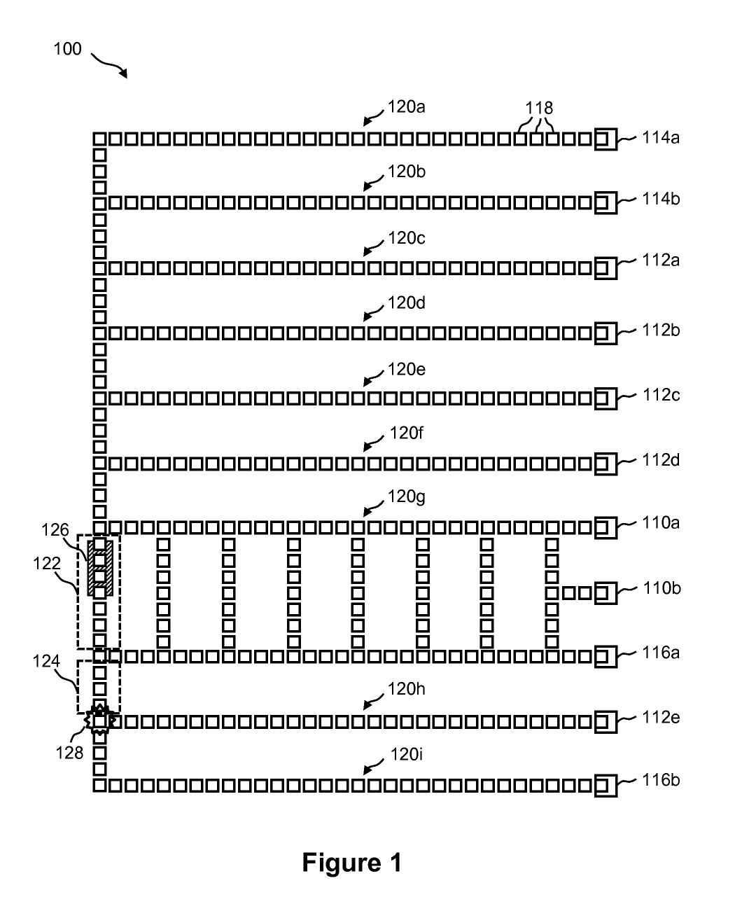

FIG. 1 illustrates a top view of an electrode arrangement 100 of an embodiment of a droplet actuator exemplifying certain aspects of the invention. The electrodes may be arranged on a substrate of a droplet actuator in a manner which is suitable for conducting droplet operations on a surface of the substrate. The substrate may be open to the atmosphere or covered. In one embodiment, the substrate is covered with a second substrate yielding a droplet operations gap between the two substrates.

Electrode lanes provide transport of nucleotide base droplets to a reactor lane. The use of dedicated lanes for nucleotide base droplets minimizes cross-contamination among nucleotides. A dedicated electrode lane provides transport of enzyme mix directly onto the detection electrode. Using a dedicated electrode lane for enzyme mix reduces enzyme deposition on the wash lanes. Reduction of enzyme contamination permits the initiation of the sequencing reaction to be precisely controlled.

Electrode arrangement 100 includes multiple dispensing electrodes, which may, for example, be allocated as sample dispensing electrodes 110a and 110b for dispensing sample fluids (e.g., DNA immobilized on magnetically responsive beads); reagent dispensing electrodes 112, i.e., reagent dispensing electrodes 112a through 112e, for dispensing different reagent fluids (e.g., dATPαs, dCTP, dGTP, dTTP, enzyme mix); wash buffer dispensing electrodes 114a and 114b for dispensing wash buffer fluids; and waste collection electrodes 116a and 116b for receiving spent reaction droplets and wash buffer. Sample dispensing electrodes 110, reagent dispensing electrodes 112, wash buffer dispensing electrodes 114, and waste collection electrodes 116 are interconnected through an arrangement, such as a path or array, of droplet operations electrodes 118. A path of droplet operations electrodes 118 extending from each dispensing and collection electrodes forms dedicated electrode lanes 120, i.e., dedicated electrode lanes 120a through 120i.

Electrode arrangement 100 may include a washing zone 122. A permanent magnet 126 is associated with wash lane 122. In the illustrated embodiment, permanent magnet 126 is located underneath wash lane 122, but it will be appreciated that a wide variety of spatial orientations is possible. Permanent magnet 126 may, in some embodiments, be embedded within the deck that holds the droplet actuator when the droplet actuator is mounted on the instrument (not shown). Permanent magnet 126 is positioned in a manner which ensures spatial immobilization of nucleic acid-attached beads during washing between the base additions. Alternative permanent magnet arrangements and arrangements making use of electromagnets will be apparent to those of skill in the art in view if this disclosure.

Electrode arrangement 100 may include a reaction zone 124. Mixing may be performed in reaction zone 124 away from permanent magnet 126. The positioning of the wash dispensing electrodes 114 and waste collection electrodes 116 improves washing efficiency and reduces time spent in washing. A detection zone 128 is positioned within or in proximity to reaction zone 124.

8.1.2 Sequencing Protocols

A variety of protocols may be executed using the droplet actuator of the invention. An example of a three-enzyme pyrosequencing protocol is as follows. A PCR amplified DNA template may be hybridized to a sequencing primer and coupled to magnetically responsive beads (or vice versa). A droplet of the beads suspended in wash buffer may be combined with a droplet of one of the four nucleotides mixed with APS and luciferin in wash buffer. A droplet containing all three enzymes (DNA polymerase, ATP sulfurylase and luciferase) may be combined with the bead and nucleotide-containing droplet. The resulting droplet may be mixed and transported to the detector location. Incorporation of the nucleotide may be detected as a luminescent signal proportional to the number of adjacent bases incorporated into the strand being synthesized, or as a background signal for a non-incorporated (mismatch) nucleotide. After the reaction is complete, the beads may be transported to the magnet and washed. Washing may, for example, be accomplished by addition and removal of wash buffer to and from the droplet while retaining substantially all beads in the droplet. Examples of suitable washing techniques are described in Pamula et al., U.S. Pat. No. 7,439,014, entitled “Droplet-Based Surface Modification and Washing,” granted on Oct. 21, 2008, the entire disclosure of which is incorporated herein by reference. This entire sequence constitutes one full pyrosequencing cycle, which may be repeated multiple times with a user defined sequence of base additions.

In a specific example, a PCR amplified DNA template hybridized to a sequencing primer may be coupled to 2.8 μm diameter magnetically responsive beads. A double-sized (800) nL droplet of the beads suspended in wash buffer may be combined with a single-sized (400 nL) droplet of one of the four nucleotides mixed with APS and luciferin in wash buffer. A single-sized (400 nL) droplet containing all three enzymes (DNA polymerase, ATP sulfurylase and luciferase) may be combined with the beads and nucleotides resulting in a quadruple-sized (1600 nL) reaction volume. The quadruple-sized droplet may be mixed and transported to the detector location. Incorporation of the nucleotide may be detected as a luminescent signal proportional to the number of adjacent bases incorporated into the strand being synthesized, or as a background signal for a non-incorporated (mismatch) nucleotide. After the reaction is complete the beads may be transported to the magnet and washed by addition and removal of wash buffer finally resulting in the 1600 nL of reaction mix being replaced by 800 nL of fresh wash buffer while essentially all of the beads may be retained in the droplet. This entire sequence constituted one full pyrosequencing cycle which may be repeated multiple times with a user defined sequence of base additions. In the above protocol, “single-sized” refers to a unit-sized droplet, which typically has a volume which is established by the size of the droplet operations electrode; a unit droplet is approximately the smallest volume that can be subjected to droplet operations based on the size of the individual electrodes. Typically, a unit sized droplet has a footprint which is approximately equal to or slightly larger than the footprint of the unit sized droplet operations electrode. The gap height, i.e., the distance between top and bottom substrates, also influences unit droplet volume.

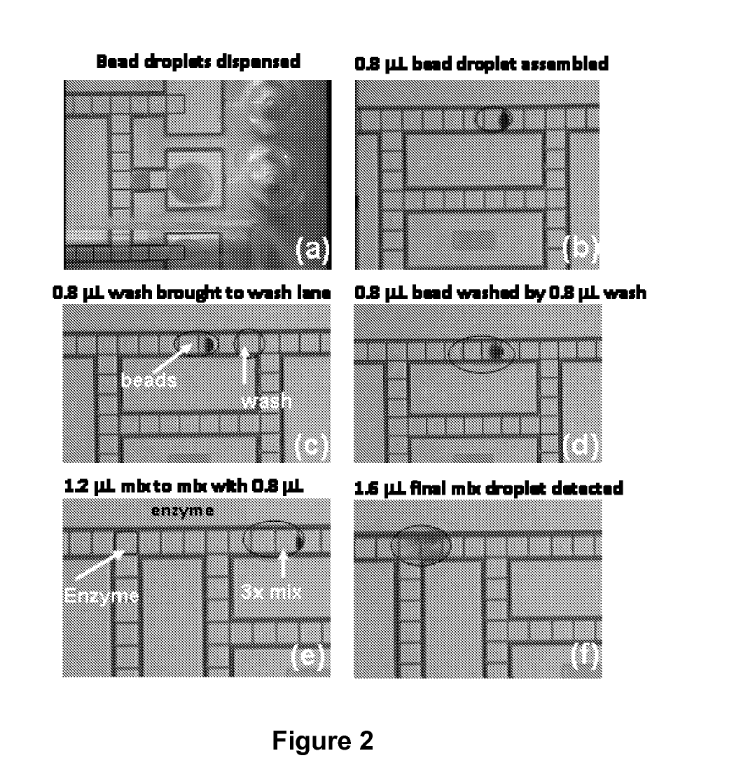

FIG. 2 illustrates steps in a process of performing a pyrosequencing reaction protocol. In one step (a), beads with the DNA-primer complex are dispensed as two 0.4 μL droplets successively from the bead reservoir. In another step (b), a 0.8 μL bead droplet is assembled on the wash lane with the beads held by the permanent magnet underneath. In another step (c and d), the 0.8 μL bead droplet is washed with a 0.8 μL wash droplet. In another step (e), a 0.4 μL enzyme droplet is dispensed and is on its way to being combined with the 1.2 μL reagent mix droplet formed by combining the 0.8 μL bead droplet and a 0.4 μL dNTP droplet dispensed from a reagent reservoir. In another step (f), the final 1.6 μL mix droplet is detected at a detection zone using a PMT mounted right above this electrode.

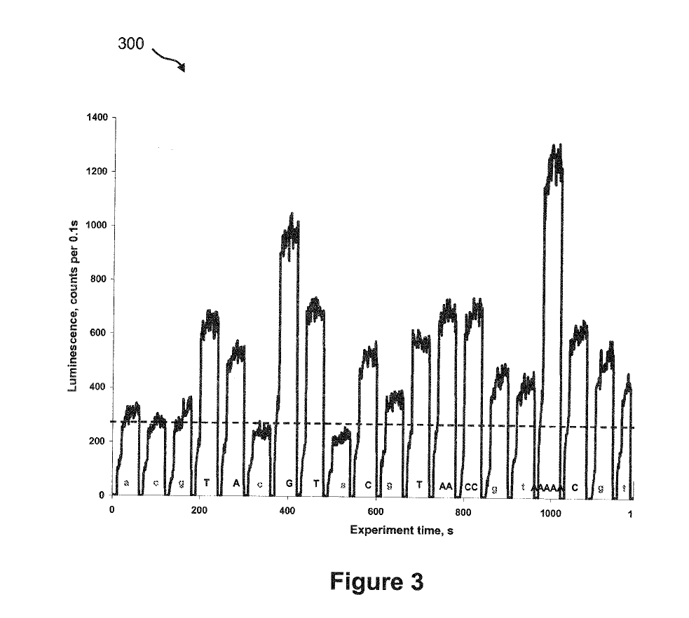

FIG. 3 shows a pyrogram 300 of on-actuator pyrosequencing results of 17-bp sequenced on a 211-bp long C. albicans DNA template using the cyclic nucleotide dispensing. FIG. 3 shows the actual pyrogram output of the experiment showing each peak. A total detection time of 60 s was used for each cycle alternating between 10 s of mixing and 10 s of detection. Non-detecting time intervals are removed from the figure for easy visualization.

Nucleic acids may be sequenced using a cyclic nucleotide dispensing strategy in which each of the four dNTP's are repeatedly added in the same order (i.e. A,C,G,T repeated in that order). Alternatively, an ordered nucleotide dispensing strategy may be used in which the order of additions is determined by a reference sequence. The order of additions proceeds according to the reference sequence until a mismatch is detected at which point additional cycles are inserted to determine the identity of the base at the mismatched position. In the absence of a suitable reference sequence, the dispensing strategy may be based on a real-time statistical analysis where the identity of the next nucleotide is predicted based on the previous results. For example, when certain genetic motifs, repeat elements or GC/AT rich regions are encountered this may reduce the total number of required dispensing cycles. In other words, if the statistical analysis of the preceding sequence suggests that one nucleotide is more likely than other nucleotides to be next in the sequence, that nucleotide will be selected first, followed by the other nucleotides in decreasing order of probability.

Wash buffer may be used as the suspending medium for reagent and enzyme mixes. The wash buffer may include salt, detergent and other constituents suitable for use in bead washing and droplet manipulation. Reagent mix may, for example, include APS, luciferin and dNTPs. Enzyme mix may, for example, include ATP sulfurylase, luciferase and DNA polymerase. Ideally a common wash buffer formulation is used for the reagent mix and enzyme mix.

In one embodiment, the wash buffer may be constituted as follows: 50 mM NaCl; 10 mM Tris-HCl; 10 mM MgCl₂; 1 mM DTT; pH 7.9. In addition to other buffer components, the buffer may include a surfactant, such as Tween-20. For example, the buffer may include less than about 1% w/w surfactant.

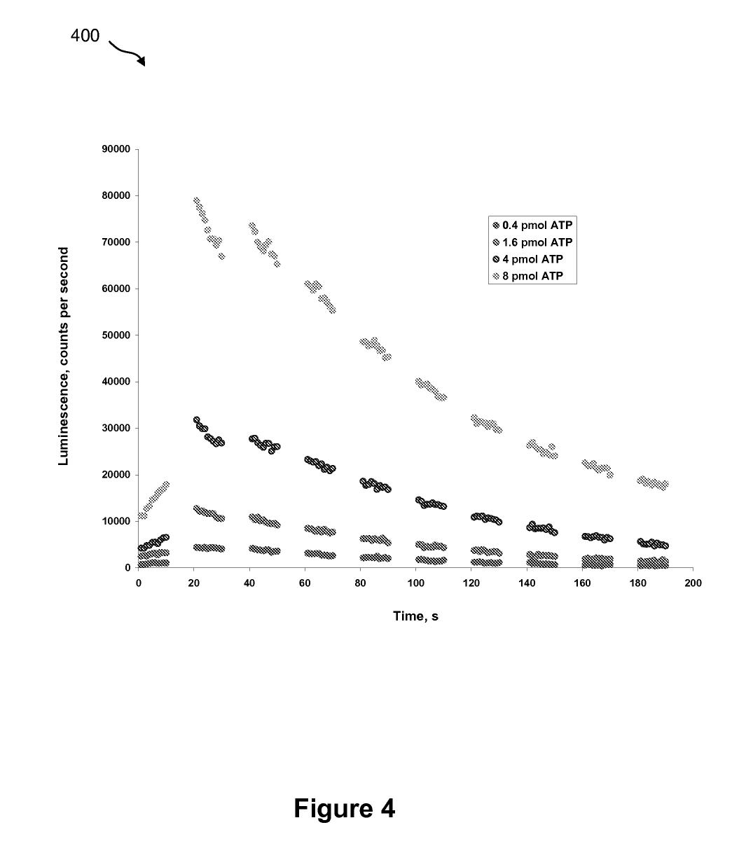

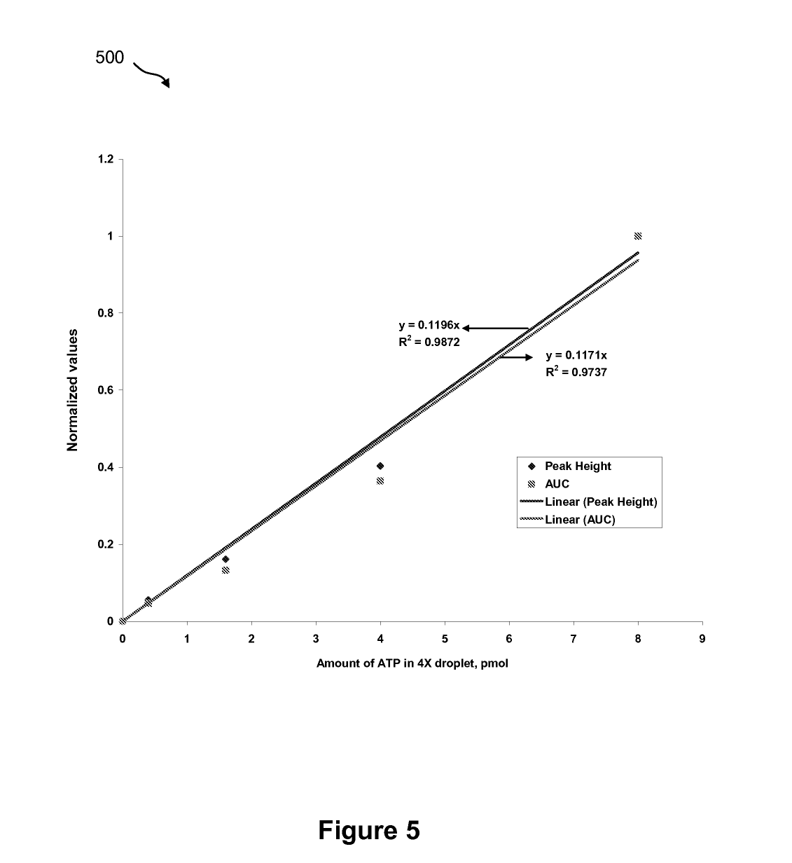

FIGS. 4 and 5 show plots of an ATP calibration performed on the system. The protocol alternates between moving the droplet for 10 s for mixing and holding it in place for 10 s to take a reading (accounting for the gaps in the curves). The data demonstrates linearity in a typical operational range. In the 3-enzyme pyrosequencing reaction protocol pyrophosphate (PPi) is generated upon successful base incorporation. PPi is converted to ATP, followed by generation of light. In order to assess the sensitivity, dynamic range, and limit of detection of the detection system, and to optimize the reagent concentrations (substrate mix, enzyme mix), we performed detection experiments for ATP standards. Solutions of four different ATP concentrations containing 200 ng/μL Luciferin were filled in four different reagent reservoirs on the same droplet actuator. Following the same on-actuator reaction protocol used for pyrosequencing, droplets from each of the reservoirs were mixed with droplets of wash and enzyme mix (540 ng/uL luciferase) and detected successively. This experiment was performed at three different settings of the diaphragm placed inside the lens assembly to restrict the amount of light entering the PMT (0.5 mm diameter aperture; 1 mm diameter aperture; no diaphragm). In FIG. 4, the results are shown for the case where the diaphragm had been removed from the optical path. As observed from the data, even at 20 fmol concentration of ATP, the peak height is still greater than 100,000 cps. Since a 200-fold reduction would be still decipherable against the background, the invention permits detection below 150 amol of ATP, or below 125 amol of ATP, or below 110 amol of ATP. This would correspond to the amount of DNA that can be put on a single bead.

Hence, in one embodiment, a nucleic acid is sequenced in a droplet having a volume that is less than about 500 pL. In another embodiment, a nucleic acid is sequenced in a droplet having a volume that is less than about 400 pL. In another embodiment, a nucleic acid is sequenced in a droplet having a volume that is less than about 300 pL. In another embodiment, a nucleic acid is sequenced in a droplet having a volume that is less than about 200 pL. In another embodiment, a nucleic acid is sequenced in a droplet having a volume that is less than about 100 pL. In another embodiment, a nucleic acid is sequenced in a droplet having a volume that is less than about 50 pL. In another embodiment, a nucleic acid is sequenced in a droplet having a volume that is less than about 25 pL. In another embodiment, a nucleic acid is sequenced in a droplet having a volume that is less than about 5 pL. In another embodiment, a nucleic acid is sequenced in a droplet having a volume that is approximately 1 pL.

In another embodiment, a nucleic acid is sequenced in a droplet having less than about 100 beads. In another embodiment, a nucleic acid is sequenced in a droplet having less than about 50 beads. In another embodiment, a nucleic acid is sequenced in a droplet having less than about 10 beads. In another embodiment, a nucleic acid is sequenced in a droplet having less than about 5 beads. In another embodiment, a nucleic acid is sequenced in a droplet having a single bead.

In another embodiment, a nucleic acid is sequenced in a droplet having 50 to about 100 beads. In another embodiment, a nucleic acid is sequenced in a droplet having 10 to 50 beads. In another embodiment, a nucleic acid is sequenced in a droplet having 5 to 10 beads. In another embodiment, a nucleic acid is sequenced in a droplet having 1 to 5 beads. In another embodiment, a nucleic acid is sequenced in a droplet having a single bead.

In still another embodiment, a nucleic acid is sequenced in a droplet having a volume that is less than about 500 pL and less than about 100 beads. In another embodiment, a nucleic acid is sequenced in a droplet having a volume that is less than about 400 pL and less than about 100 beads. In another embodiment, a nucleic acid is sequenced in a droplet having a volume that is less than about 300 pL and less than about 100 beads. In another embodiment, a nucleic acid is sequenced in a droplet having a volume that is less than about 200 pL and less than about 100 beads. In another embodiment, a nucleic acid is sequenced in a droplet having a volume that is less than about 100 pL and less than about 50 beads.

In another embodiment, a nucleic acid is sequenced in a droplet having a volume that is less than about 100 pL and less than about 50 beads. In another embodiment, a nucleic acid is sequenced in a droplet having a volume that is less than about 100 pL and less than about 25 beads. In another embodiment, a nucleic acid is sequenced in a droplet having a volume that is less than about 100 pL and less than about 5 beads.

In another embodiment, a nucleic acid is sequenced in a droplet having a volume that is less than about 50 pL and less than about 50 beads. In another embodiment, a nucleic acid is sequenced in a droplet having a volume that is less than about 50 pL and less than about 25 beads. In another embodiment, a nucleic acid is sequenced in a droplet having a volume that is less than about 50 pL and less than about 5 beads.

In another embodiment, a nucleic acid is sequenced in a droplet having a volume that is less than about 10 pL and less than about 10 beads. In another embodiment, a nucleic acid is sequenced in a droplet having a volume that is less than about 10 pL and less than about 5 beads. In another embodiment, a nucleic acid is sequenced in a droplet having a volume that is less than about 5 pL and less than about 5 beads. In another embodiment, a nucleic acid is sequenced in a droplet having a volume that is less than about 5 pL and 1 or 2 beads. In another embodiment, a nucleic acid is sequenced in a droplet having a volume that is about 1 pL and about 1 bead.

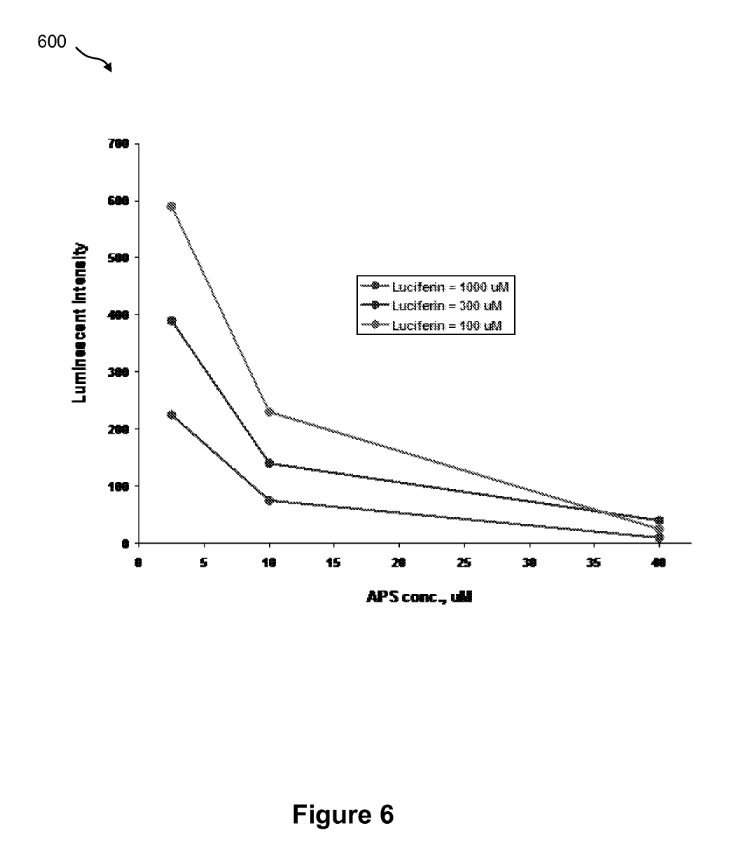

FIG. 6 shows a plot 600 of the dependence of chemiluminescent signal intensity on the concentration of substrates used to convert PPi to light. The experiment was performed by mixing 1 μM of PPi with the substrates APS and luciferin along with the enzymes luciferase and ATP sulfurylase in a total volume of 50 μL. At high concentrations, APS and luciferin inhibit the chemiluminescent signal generation (FIG. 6). But at lower concentrations, the signal corresponding to the homopolymer runs is not quite proportional. In various embodiments, APS concentration in the final pyrosequencing droplet subjected to detection (e.g., see panel (f) in FIG. 2) ranges from about 1 to about 20 μM APS, or from about 5 to about 15 μM APS, or from about 8 to about 12 μM APS. In various embodiments, luciferin concentration in the final pyrosequencing droplet (e.g., see panel (f) in FIG. 2) ranges from about 25 to about 75 ng/μL luciferin, or from about 35 to about 65 ng/μL luciferin, or from about 45 to about 55 ng/μL luciferin. In one specific embodiment, concentrations are 10 μM APS and 50 ng/μL luciferin in the final 1.6 μL droplet. It will be appreciated that starting concentrations of these reagents may be varied depending on the specific protocol employed in order to achieve the final concentrations described here.

The ability to achieve longer read lengths and to read homopolymer runs with high fidelity in pyrosequencing is dependent on the stability and specificity of the catalytic activity of the DNA polymerase while accomplishing complete base incorporations. Three different DNA polymerases were tried—BST polymerase, Klenow exo- and Sequenase 2.0. While BST polymerase exhibits strong binding to DNA and stability against washing, it requires higher temperatures (˜60° C.) for optimal activity. Sequenase 2.0, commonly used for dideoxy Sanger sequencing, is also known to be stable for sequencing several hundreds of bases, but most of the commercial preparations of Sequenase 2.0 have a strong PPi background requiring multiple purification steps before use for pyrosequencing. Klenow exo- has good thermal stability and sequencing specificity. The reaction protocol was optimized to supply fresh Klenow at 0.7 U/μL during every new base addition since it is slightly susceptible to unbind from the DNA during washing.

In one embodiment, the methods of the invention make use of a polymerase preparation having low PPi background. For example, the PPi background may be sufficiently low to permit detection of PPi released during a nucleotide incorporation event with statistically reliable results. Similarly, the PPi background may be sufficiently low to permit detection of PPi released during a nucleotide incorporation event with diagnostically acceptable precision and/or accuracy.

The chemiluminescent signal generated may be a flash (strong peak over a short period of time). The chemiluminescent signal generated may be a glow (moderate signal intensity over extended period of time). A flash system is preferable for lower detection levels of PPi and for faster detection in high throughput sequencing. The flash technique requires concentrations of luciferase and sulfurylase that are sufficiently high to produce the flash.

In an alternative embodiment, sulfurylase and/or luciferase may be coupled to magnetically responsive beads in separate droplets localized on the droplet actuator, such as in the droplet operations gap or in a reservoir in fluid communication with the droplet operations gap. Because sulfurylase and luciferase are separated, the regeneration cycle of pyrophosphate to ATP is disrupted, and the throughput of the assay is increased. In this example:
1. A template droplet may be provided with PCR amplified DNA template hybridized to a sequencing primer may be coupled to a third group of magnetically responsive beads.
2. A sulfurylase droplet may be provided with sulfurylase, the second enzyme in the pyrosequencing reaction, coupled to magnetically responsive beads.
3. A luciferase droplet may be provided with luciferase, the third enzyme in the pyrosequencing reaction, coupled to magnetically responsive beads.

The template droplet may be combined with a droplet including one of the four nucleotides and pyrosequencing reagents (e.g., DNA polymerase, APS and luciferin in wash buffer) to yield a reaction droplet in which the pyrosequencing reaction (i.e., incorporation of dNTP by DNA polymerase). Supernatant from this reaction may be removed and combined with the sulfurylase droplet. After a sufficient period of time for conversion of pyrophosphate to ATP, supernatant from the sulfurylase droplet may be removed and combined with the luciferase bead droplet for generation of a luminescent signal and detection.

The second enzymatic reaction in a pyrosequencing protocol typically includes enzymatic conversion of pyrophosphate to ATP using sulfurylase and APS as a substrate. Because luciferase is typically used in the third enzymatic reaction of a pyrosequencing protocol, there is potential for generation of relatively high background luminescence due to the luciferase-APS interaction. An alternative method for conversion of pyrophosphate to ATP includes the use of the enzyme pyruvate orthophosphate dikinase (PPDK) and substrates AMP and phosphoenolpyruvate. Because AMP and phosphoenolpyruvate are inactive for the luciferase-catalyzed reaction that generates a high background luminescence, reduced background signals and increased sensitivity (e.g., significantly reduced amount of input sample) in a pyrosequencing reaction may be achieved.

In one embodiment, the invention provides a multiplexed pyrosequencing with detection at a single spatial location. ATP or pyrophosphate droplets from different simultaneously run pyrosequencing reactions can be sequentially assayed at the common detection electrode. An example of such a protocol is as follows: (1) 2 droplets of DNA-beads, are transported to an edge of the magnet, combined and held there; (2) these 2× bead droplets are then washed with 2× wash droplet (assembled from two 1× droplets) for 8 cycles; (3) the 2× bead droplet is then transported away from the magnet; (4) a 1× dNTP droplet and 1× enzyme droplet (Klenow polymerase) are then added sequentially from the respective reagent reservoirs to the beads and the distribution grid is washed with 1× wash droplets; (5) the 4× mix droplet is then shuttled back and forth on top of the magnet on 3 electrodes for about 40 sec and then parked on an edge of the magnet; (6) the 4× droplet is split into two 2× at the edge of the magnet (1 containing PPi and another containing beads); (7) the PPi droplets from all the lanes are then moved to the assembly electrodes and are detected sequentially; (8) a 1× enzyme droplet (PPi detection) from the enzyme reservoir is transported to the detection spot, and while holding that droplet, the 2× PPi droplet is then transported to the detection electrode, combined with the enzyme droplet and the 3× droplet shuttled as a 2× (with scrunch) for about 16 sec and then detected; (9) the detection is done for 10 sec with 200 ms integration time (0 samples); (10) four 1× wash droplets are dispensed from the secondary wash reservoir are then transported across the detection spot to the waste, thus cleaning the spot thoroughly; (11) steps 8, 9 and 10 are repeated for the next 3 PPi droplets from other lanes; and (12) steps 3-11 are repeated for the next dNTP, and the process is continued till the entire sequence is complete.

All the reagents for pyrosequencing (dNTPs, enzymes and substrates) can be cleaned up enzymatically to remove any ATP or pyrophosphate contamination. Pyrophosphatase may be used for cleaning up PPi. Apyrase may be used for cleaning up ATP. Cleaner reagents produce better data quality and may contribute to longer sequencing reads. As an example, dNTPs undergo hydrolysis when stored, to form phosphates and pyrophosphates. This hydrolysis contributes to background counts. The presence of ATP in water and other buffers used to constitute the sample, substrate and enzyme solutions also contribute to the background counts in pyrosequencing. Pyrophosphatase (PPiase) attached to M270 Dynal beads (with carboxylic functional groups) can be used to cleanse the PPi in the solutions.

Linear dependence of signal to concentration for PPi may be important for obtaining proportional signals in homopolymer sequencing. The regeneration of pyrophosphate in PPi assay may be detrimental to obtaining linearity. In one embodiment, the sequencing assay further includes separating the PPi-to-light assay into PPi-to-ATP and ATP-to-light steps. This separation may be accomplished spatially or temporally. Temporally, the PPi regeneration can be delayed by accelerating the first step of ATP generation relative to the second step, by increasing the concentration of ATP sulfurylase and/or limiting the concentration of adenosine phosphosulfate (APS). Spatially, the ATP sulfurylase can be attached to beads and the ATP generation and ATP detection can be decoupled spatially using magnets to retain the ATP sulfurylase beads. To date, the inventors have demonstrated spatial sequestering over a range of PPi signal, 0-12 uM equivalent to up to 20 bp signal.

8.1.3 Remedial Measures for PPi Contamination in PCB

The inventors have discovered that PPi contamination on the PCB droplet actuator materials and chemical reagents may in some cases contribute to high background, significantly limiting the sensitivity that can be obtained. Pyrophosphates are commonly used in the printed circuit board industry. Baths of copper pyrophosphate are used to electroplate PCBs and melamine pyrophosphate is used as a flame retardant in materials such as adhesives and polymers used in the PCB industry.

In one embodiment, the invention includes PCB chips in which remedial measures have been used to reduce PPi contamination or to reduce interference caused by PPi contamination. Remedial measures may reduce PPi contamination sufficiently to eliminate undue interference of background PPi with detection of PPi generated by the sequencing reaction. A PCB material may be selected which is manufactured without a pyrophosphate treatment or with a reduced treatment sufficient to eliminate undue interference of background PPi from the PCB with detection of PPi generated by the sequencing reaction. The PCB may be subjected to procedures in the droplet actuator manufacturing process to reduce the introduction of PPi contamination. The PCB may be washed or otherwise treated to reduce PPi. The PCB may be washed in an acid bath to reduce PPi contamination. The PCB may be treated with an enzyme, such as pyrophosphatase to reduce PPi contamination. The PCB may be coated with a substance that blocks PPi release during a sequencing protocol. For example, the PCB may be coated with a CYTOP® surface coating having a thickness sufficient to eliminate undue interference of background PPi from the PCB with detection of PPi generated by the sequencing reaction.

In one embodiment, the PCB substrate is coated with a thick fluoropolymer coating, such as a CYTOP® coating. The fluoropolymer coating may have a thickness which is sufficient to reduce PPi contamination to an acceptable level, such as a diagnostically acceptable level. For example, the fluoropolymer coating may have a thickness which is greater than about 200 nm. The fluoropolymer coating may have a thickness which is greater than about 500 nm. The fluoropolymer coating may have a thickness which is greater than about 1 μm. The fluoropolymer coating may have a thickness which is greater than about 1.5 μm. The fluoropolymer coating may have a thickness which is greater than about 2 μm. The fluoropolymer coating may have a thickness which ranges from about 0.5 to about 5 μm. The fluoropolymer coating may have a thickness which ranges from about 1 to about 3 μm.

In another embodiment, the inventors have found that spray coating of a fluoropolymer, such as CYTOP® coating, is superior to dip coating for preventing PPi leaching. During dip coating, PPi can leach into the polymer bath, e.g., into the CYTOP® coating bath. However, when the PCB is spray coated, the polymer mist covers the polyimide surface of the chips and contains the underlying PPi. Thus, in one embodiment, the invention provides for conducting a pyrosequencing reaction on a PCB chip that has been spray coated with a polymer coating, such as a fluoropolymer coating, such as a CYTOP® coating. In this manner, background signal caused by PPi contamination of the PCB may be substantially reduced or even eliminated.

In various embodiments, the invention provides for remedial measures which reduce PPi background by at least 75% relative to background in the absence of the remedial measure. In various embodiments, the invention provides for remedial measures which reduce PPi background by at least 85% relative to background in the absence of the remedial measure. In various embodiments, the invention provides for remedial measures which reduce PPi background by at least 95% relative to background in the absence of the remedial measure. In various embodiments, the invention provides for remedial measures which reduce PPi background by at least 99% relative to background in the absence of the remedial measure. In various embodiments, the invention provides for remedial measures which substantially eliminate PPi background.

In various embodiments, the invention provides for applications of coatings of sufficient thickness to reduce PPi background by at least 75% relative to background in the absence of the coating. In various embodiments, the invention provides for applications of coatings of sufficient thickness to reduce PPi background by at least 85% relative to background in the absence of the coating. In various embodiments, the invention provides for applications of coatings of sufficient thickness to reduce PPi background by at least 95% relative to background in the absence of the coating. In various embodiments, the invention provides for applications of coatings of sufficient thickness to reduce PPi background by at least 99% relative to background in the absence of the coating. In various embodiments, the invention provides for applications of coatings of sufficient thickness to substantially eliminate PPi background. The PPi background reduction or elimination may be achieved without eliminating the capability of the droplet actuator to conduct droplet operations.

Droplet transport pathways or reaction sites or detection sites may be washed as part of an assay protocol to remove PPi from the droplet actuator surfaces. One or more wash droplets may be transported through the pathway or reaction site or detection site prior to introduction of a sample droplet for sequencing. The wash droplet(s) may include any solution which chemically modifies, inactivates, absorbs or otherwise removes the PPi. For example, the wash droplet(s) may include pyrophosphatase or pyrophosphatase beads. The inventors have discovered that circulating a sufficient number of wash droplets across electrodes before executing a pyrosequencing protocol reduces background PPi to the basal level. For example, the number of wash droplets required may be 1, 2, 3, 4, 5, 6, 7, 8, 9, 10 or more. Ideally, all electrodes used in the protocol are subjected to washing. A variety of buffer compositions may be used. In one embodiment, the buffer included Tris acetate, EDTA, Mg acetate, NaCl, Tween-20, DTT, and water. For example, in a specific embodiment, the buffer may include 50 mM Tris acetate, 10 mM EDTA, 25 mM Mg acetate, 50 mM NaCl, 0.01% Tween, 1 mM DTT, and water. The residence time of the wash droplet on the electrodes being washed is also an important factor in assuring substantially complete washing. Higher droplet speeds require a greater number of droplets to achieve a reduction in background PPi that is similar to fewer droplets residing on the electrodes for longer periods.

On-actuator pyrophosphatase beads may be prepared using various techniques for coupling pyrophosphatase to beads without eliminating the pyrophosphatase activity. In one example, 100 μL of 1 mg/mL (100 μg) pyrophosphatase (Sigma Cat #: I 5907) was buffer exchanged into 0.1 M sodium phosphate, 0.15M sodium chloride, pH 7.2 (PBS), using Zeba Spin Columns 0.5 ml (Pierce Cat #: 89882). 100 μL (3 mg) of Dynabeads M-270 Carboxylic Acid was pipetted into a tube and washed three times with 500 μL 25 mM MES, pH 5.0. The beads were then incubated in 50 μL of 0.26 M 1-ethyl-3-(3-dimethylaminopropyl)carbodiimide hydrochloride (EDC) and 50 μL of 0.43 M N-hydroxy sulfosuccinimide (sulfo-NHS) for 30 min at room temperature. The beads were held by a magnet and the supernatant was removed. The activated beads were then washed with 500 μL 25 mM MES, pH 5.0 buffer and then finally with the PBS buffer, pH 7.2. 100 μL of desalted pyrophosphatase in PBS pH 7.2 was added to the beads and incubated at room temperature for 30 min. The beads were then brought down by the magnet and then resuspended in 300 vL 0.05M ethanolamine in PBS, pH 8.0 quench buffer. After incubating for 1 hr, the beads were removed from quench buffer, washed four times in 100 μL PBS pH 7.2 and then stored in the same at 4° C. The pyrophosphatase beads were typically resuspended in pyro wash buffer before use. Droplets including pyrophosphatase beads may be transported onto electrodes within a detection window to eliminate contaminating PPi before and/or after transport of a pyrosequencing reaction droplet onto the same electrodes for detection. In some case, to enhance cleaning, the droplet may be transported back and forth or otherwise subjected to agitation using droplet operations or other agitation means in the presence of the detection window.

As an example, the inventors have observed that the final mix (8 uL) on the plate reader (4 uL of wash+2 uL of enzyme mix+2 uL of dNTP & substrate mix) gave about 100,000 counts for area under the curve for 1 min of light collection (‘basal’ counts). On the droplet actuator, the same combination at 1.6 uL volume gave the same 100,000 counts. In some experiments with new dNTPs and those that were cleaned with pyrophosphatase beads, where residual PPi in the reagents was degraded, t the mix gave 50,000 counts.

The inventors' experiments related to background reduction on the chip surface typically started off with 400,000 counts and was reduced to either 200,000 (50% reduction) or 100,000 (75% reduction) after treatment. If background caused by PPi contamination in the reagents is not considered, then 75% treatment would actually suggest approximately 100% reduction.

Generally speaking, the reduction in background can be measured based on signal count. The inventors performed independent PPi and ATP calibrations on the chip, and determined for example that 0.1 pmol (or 100 fmol) of PPi corresponds to about 100,000 counts at collection for 1 min with 1 mm diaphragm aperture on chip and for 100 ms integration time. Under these conditions, background reduced to from about 50 to about 100 fmol of PPi. The reagent mix itself often included almost 50-80 fmol of PPi.

8.1.4 Beads and Washing

In various sequencing embodiments, as reservoir as in amplification embodiments, the invention makes use of magnetically responsive beads. Magnetically responsive beads may be used as a solid phase for attachment of the nucleic acid. Magnetically responsive beads can be conveniently manipulated within droplets in a digital microfluidic system. Washing is accomplished by transporting a bead-containing droplet to a position on the droplet actuator located directly above a permanent magnet. Wash droplets are then merged with the bead-containing droplet on one side and supernatant removed from the opposite side by splitting-off a portion of the combined droplet. Magnetically responsive beads may be washed without significant loss of beads. Displacement washing allows a “wash through” process to occur without subjecting the beads to a wash droplet's surface tension boundary. The beads may be, for example, provided in a single-sized droplet. A wash-through double-sized or greater droplet is transported through the bead droplet, and mixing causes dilution washing. The process continues with fresh double-sized wash droplets until complete. Of course, the starting droplet may be single-sized or greater, and the wash-through droplet is simply greater in volume relative to the starting droplet, typically the volume of the wash-through droplet is at least two times the size of the starting droplet. Examples of suitable washing techniques are described in Pamula et al., U.S. Pat. No. 7,439,014, entitled “Droplet-Based Surface Modification and Washing,” granted on Oct. 21, 2008, the entire disclosure of which is incorporated herein by reference.

Beads may be prepared using a variety of techniques for binding nucleic acid to the beads. In one example, beads are prepared as follows: 50 μL of Streptavidin M280 Dynabeads (Invitrogen) were washed in binding buffer (10 mM Tris-HCl, pH 7.6, 2 M NaCl, 1 mM EDTA, 0.1% Tween 20) three times and resuspended in a final volume of 50 μL. 10 μg of PCR product was added to the beads. Beads and DNA were incubated at 65° C. for 15 min with periodic mixing. The DNA was made single-stranded by incubating beads in 100 μl of 0.5 M NaOH for 1 min. The beads were washed in NaOH one time and then 3 times in Mag-Annealing buffer (20 mM Tris-Acetate pH 7.6, 5 mM Mg-Acetate) and resuspended in a final 50 μl volume.

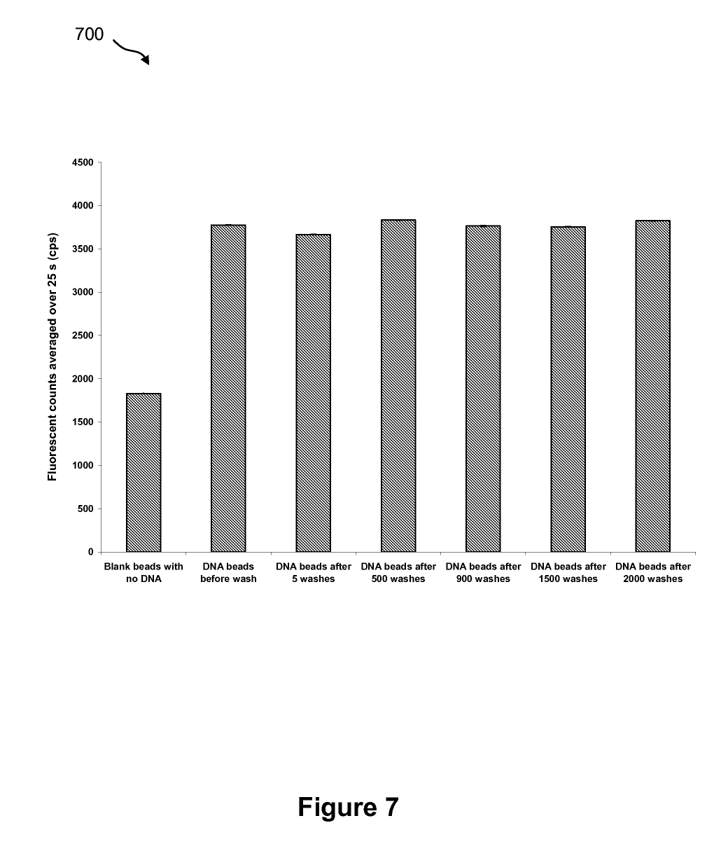

FIG. 7 shows a plot 700 of fluorescence of the FAM-labeled primer/DNA attached to beads monitored with washes. The data show that the DNA template/primers are strongly bound to the beads and do not unbind during washing. A 19-bp biotinylated FAM-labeled primer was hybridized to a 40-bp C. albicans DNA template and the complex was attached to streptavidin-coated M280 beads. Eight μg of such beads were encapsulated in an 800 nL droplet and held at a defined location on the droplet actuator using a permanent magnet placed underneath. The beads were washed subsequently for 2000 cycles with 800 nL droplets of wash buffer and the fluorescence of the beads was monitored before and after the 2000 wash cycles. From FIG. 7, it can be seen that the fluorescence of the beads is not reduced even after washing them for 2000 cycles, thus demonstrating the stability criterion for long sequencing runs. Also evident from this demonstration are the ability of the current droplet actuator architecture of the inventors and the robustness of the CYTOP® surface coating in continuously dispensing 2000 cycles of wash buffer from the wash reservoir and discarding them to the waste reservoir.

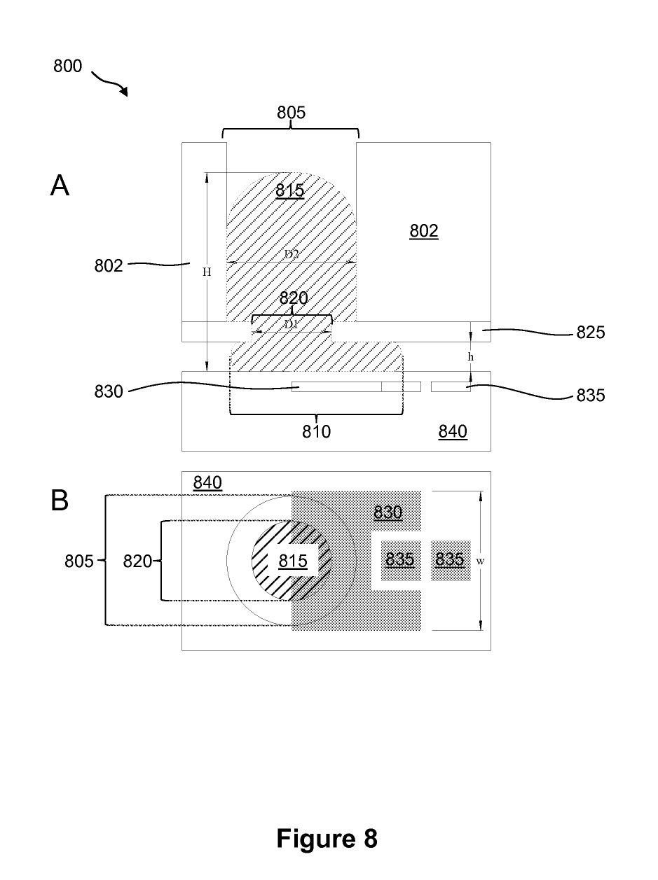

Uninterrupted pyrosequencing runs require that many thousands of droplets can be dispensed rapidly without reloading of droplet actuator reservoirs. In order to achieve this desired level of throughput the inventors have developed devices for formation of large numbers of droplets that can be dispensed on droplet actuator. FIG. 8 illustrates top and side views of a high-capacity reservoir design 800 incorporating a reservoir assembly 802 including a reservoir 805 positioned above the on-droplet actuator reservoir 810 to provide a constant liquid feed. The illustrated interface allows reagent inputs from microliters to milliliters. Wash and waste reservoirs enable “load and go” continuous droplet actuator operation. Reservoir 805 continually feeds liquid 815 into the chamber through opening 820 in top substrate 825 which brings liquid 815 into contact with the on-droplet actuator dispensing apparatus, which includes reservoir electrode 830, and droplet operations electrodes 835 associated with bottom substrate 840. A wide range of reservoir-types may be provided to accommodate application-specific reagent volume requirements. For example, in one embodiment, sample wells may be configured to dispense 20 droplets (about 320 nL each) from an initial approximately 20 μL sample. As another example, wash wells may be configured to produce thousands of wash droplets, e.g., one configuration produces over 2300 wash droplets (320 nL) from a 3.5 mL starting volume. In one embodiment, the wells are provided in an open format for loading by a user. In another embodiment, the wells are provided in a closed format, e.g., to maintain sterility. The user may, for example, remove a cover prior to loading or may, as another example, inject liquid through a cover into the reservoir. In yet another embodiment, one or more reservoirs is pre-loaded with a reagent or buffer.

8.1.5 Alternative Droplet Actuator Architectures for Sequencing

Referring again to FIG. 1, an electrode arrangement for pyrosequencing includes dedicated electrode lanes for dispensing, storing and transporting reagent fluids (e.g., dATP, dTTP, dCTP, dGTP, enzyme mix, and substrate) and wash buffer fluids. In this arrangement, a common electrode lane is used to transport reagent fluid droplets and wash buffer droplets from the dedicated electrode lanes to common reaction and detection zones. Because a common electrode lane is used, there is potential for cross-contamination between reagent fluid droplets during the pyrosequencing reaction. Further, the electrode arrangement is such that reagent fluid droplets and wash buffer droplets are transported over a relatively large number of droplet operations electrodes to the reaction and detection zones. Because of the transport distance from dedicated electrode lanes to reaction zones, there is the potential for decreased time-to-result (throughput) in the assay.

Alternative electrode arrangements for pyrosequencing on a droplet actuator may be used to increase throughput and minimize cross-contamination between droplets. In various embodiments, droplet operations electrodes (i.e., dedicated electrode lanes) are organized into unit cells. For example, separate dedicated electrode lanes may be used for dispensing and storing reagent droplets (e.g., dNTP reaction droplets), washing and waste collection. In the unit cell configuration, the number of droplet operation electrodes interconnecting dispensing and collection electrodes to reaction and washing zones is minimized. The configuration of a unit cell is optimized such that all steps in a sequencing protocol may be performed within the unit cell.

In one embodiment, dedicated electrode lanes are configured to provide transport of nucleotide base reagent droplets to a single reaction electrode and detection zone (i.e., a single reaction zone).

In another embodiment, dedicated electrode lanes are configured to provide transport of nucleotide base reagent droplets to individual reaction electrodes and detection zones arranged in a circular array (i.e., four separate reaction zones). The unit cell may be configured to permit reaction droplets movement in a clockwork fashion, i.e., clockwise and/or counterclockwise.

In yet another embodiment, a magnet or other bead retention mechanism is used to transport a sample droplet that includes beads or other structures to which nucleic acid template is bound around a circular array of droplet operations electrodes that is configured for pyrosequencing.

In yet another embodiment, a sample droplet that contains magnetically responsive beads may be immobilized in a capillary device and slugs of reagent and wash fluids sequentially moved across the immobilized sample droplet.

In yet another embodiment, DNA-primer complexes are bound to one or more magnetically responsive beads that are immobilized on a centrifugal microfluidic device such as a compact disc (CD; e.g., LabCD type device). Centrifugal force is used to provide a constant, sequential supply of fresh reagent fluids and wash buffer fluids from multiple dispensing channels over the immobilized bead.

In yet another embodiment, throughput (time-to-result) of pyrosequencing on a droplet actuator may be increased by implementing a “look-ahead-sequencing” protocol.

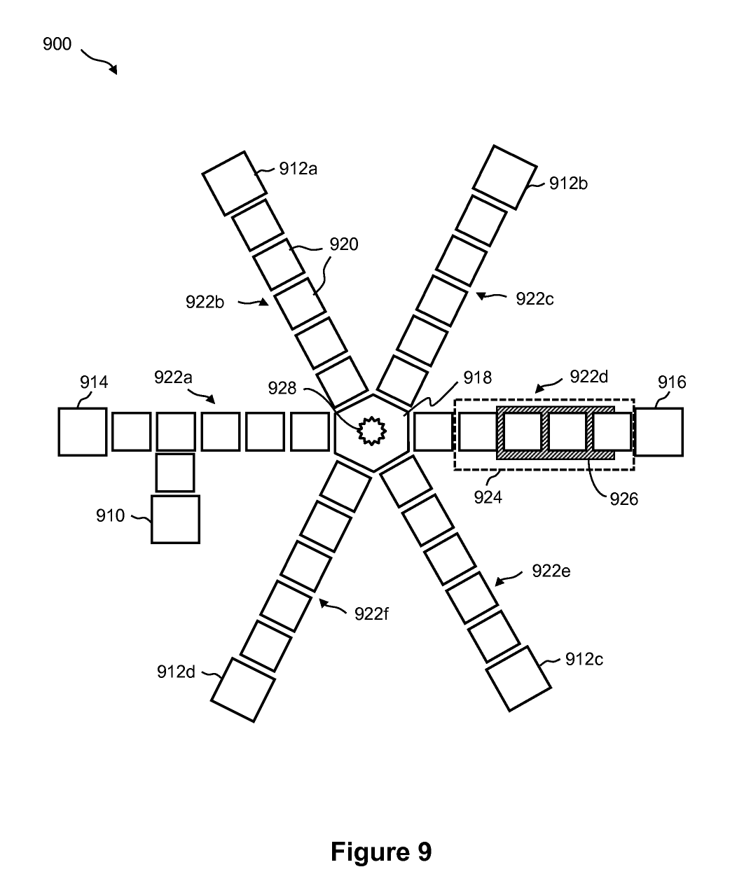

FIG. 9 illustrates a top view of an electrode arrangement 900 of a droplet actuator organized into a unit cell that includes a single reaction zone. In this embodiment, four dedicated electrode lanes provide transport of nucleotide base droplets (i.e., one dedicated electrode lane for each dATP, dTTP, dCTP and dGTP reagent droplets) to a single reaction and detection electrode. In one example, reagent droplets may include enzyme mix and detection substrate. Because certain electrode lanes are dedicated to dispensing specific reagent fluids and/or wash buffers, reagent droplets and/or wash buffer droplets may be dispensed and stored in the respective dedicated electrode lanes to increase throughput in the pyrosequencing reaction.

Electrode arrangement 900 includes multiple dispensing electrodes, which may, for example, be allocated as a sample dispensing electrode 910 for dispensing sample fluid (e.g., DNA/primer immobilized on magnetically responsive beads); reagent dispensing electrodes 912, i.e., reagent dispensing electrodes 912a through 912d, for dispensing different reagent fluids (e.g., one of the four dNTPs, enzyme mix, APS, luciferin); wash buffer dispensing electrode 914 for dispensing wash buffer fluids; and waste collection electrode 916 for receiving spent reaction droplets. Sample dispensing electrode 910, reagent dispensing electrodes 912, wash buffer dispensing electrode 914, and waste collection electrode 916 are connected to a single reaction electrode 918 through an arrangement, such as a path or array, of droplet operations electrodes 920. A path of droplet operations electrodes 920 extending from each dispensing and collection electrodes forms dedicated electrode lanes 922, i.e., dedicated electrode lanes 922a through 922f. In the illustrated embodiment, the electrode lanes are radially arranged with respect to detection zone 928, but it will be appreciated that other embodiments are possible within the scope of the invention. For example, as illustrated, the lanes are generally linear and straight, but it will be appreciated that the lanes may be curvilinear or otherwise include changes in the direction or linearity of droplet transport. For example, in another embodiment, all reservoirs may be at a common edge of the droplet actuator, and may nevertheless converge on a detection zone.

Electrode arrangement 900 may include a washing zone 924. A permanent magnet 926 may be located underneath wash zone 924. Permanent magnet 926 may be embedded within the deck that holds the droplet actuator when the droplet actuator is mounted on the instrument (not shown). Permanent magnet 926 is positioned in a manner which ensures spatial immobilization of nucleic acid-attached beads during washing between the base additions. Mixing may be performed on reaction electrode 918 away from permanent magnet 926. The positioning of the wash buffer dispensing electrode 914 and waste collection electrode 916 improves washing efficiency and reduces time spent in washing. Detection zone 928 is positioned in proximity of reaction electrode 918.

In operation, a sample droplet (not shown) may be dispensed from sample dispensing reservoir 910 onto dedicated electrode lane 922a and transported using droplet operations to reaction electrode 918. A reagent droplet (not shown) may, for example, be dispensed from reagent reservoir 912a onto dedicated electrode lane 922b and combined with the sample droplet at reaction electrode 918 to yield a reaction droplet. Incorporation of the nucleotide may be detected as a luminescent signal. After the reaction is complete, the reaction droplet may be transported to washing zone 924 and washed by addition and removal of wash buffer droplets dispensed from dedicated electrode lane 922a. The reaction droplet may then be transported back to reaction electrode 918 for a second cycle of pyrosequencing (dNTP incorporation and detection). Any number of sequencing cycles may be performed with a user defined sequence of base additions. In other embodiments, sample capture and washing may also be performed on the electrode arrangement.

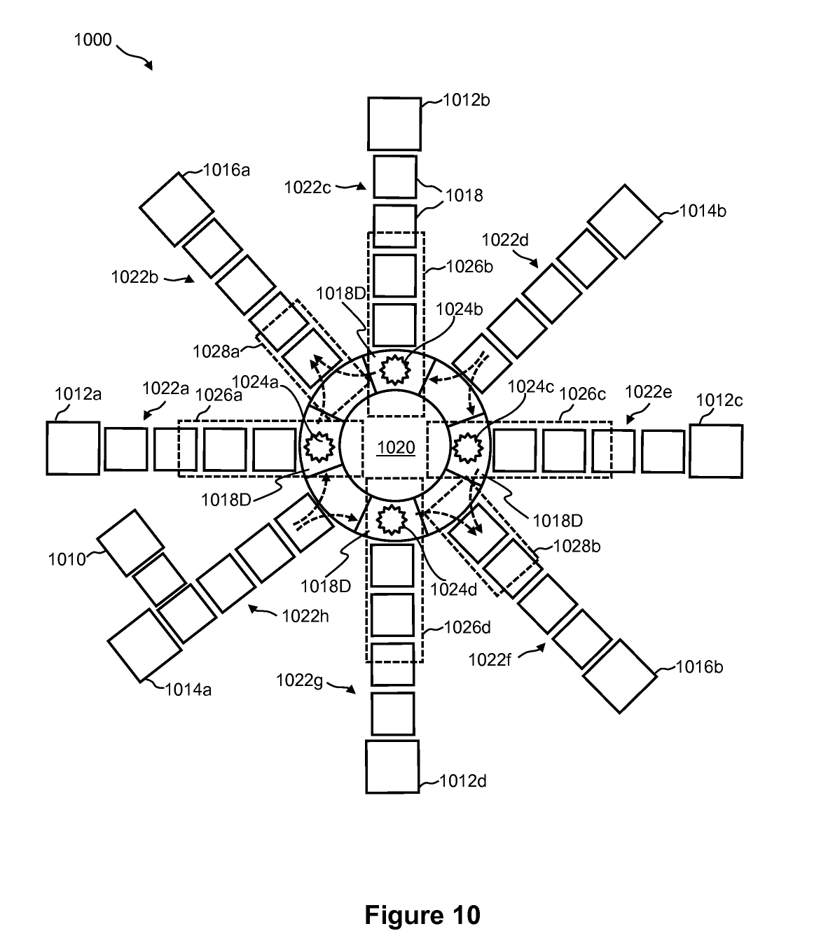

FIG. 10 illustrates a top view of an electrode arrangement 1000 of a droplet actuator organized into a unit cell that includes four separate reaction zones. In this embodiment, dedicated electrode lanes for dispensing and storing each dNTP (i.e., dATP, dTTP, dCTP and dGTP) are aligned with a circular array of droplet operations electrodes to form individual reaction zones with separate detection zones. Dedicated electrode lanes for dispensing wash buffer droplets and washing operations are interspersed among the individual reaction zones.

Electrode arrangement 1000 includes multiple dispensing electrodes, which may, for example, be allocated as a sample dispensing electrode 1010 for dispensing sample fluid (e.g., DNA immobilized on magnetically responsive beads); reagent dispensing electrodes 1012, i.e., reagent dispensing electrodes 1012a through 1012d, for dispensing different reagent fluids (e.g., dATPαs, dTTP, dCTP, dGTP, enzyme mix, substrate); wash buffer dispensing electrodes 1014a and 1014b for dispensing wash buffer fluids; and waste collection electrodes 1016a and 1016b for receiving spent reaction droplets. Sample dispensing electrode 1010, reagent dispensing electrodes 1012, wash buffer dispensing electrodes 1014, and waste collection electrodes 1016 are interconnected through an arrangement, such as a path or array, of droplet operations electrodes 1018. Certain droplet operations electrodes 1018 may be arranged to form a circular array 1020 of droplet operations electrodes. A path of droplet operations electrodes 1018 extending from each dispensing and collection electrode connects the dispensing and collection electrodes to circular array 1020. The path of droplet operations electrodes 1018 extending from each dispensing and collection electrode forms dedicated electrode lanes 1022, i.e., dedicated electrode lanes 1022a through 1022h.

Electrode arrangement 1000 may include one or more detection zones or spots 1024. In one example, four detection zones 1024 (e.g., detection zones 1024a through 1024d) are positioned in proximity to certain droplet operations electrodes 1018 (e.g., 1018D) in circular array 1020. In this example, detection zones 1024 are positioned on droplet operations electrodes 1018D where dedicated electrode lanes 1022a, 1022c, 1022e, and 1022g connect with certain droplet operations electrodes 1018D in circular array 1020. The arrangement of dedicated electrode lanes 1022a, 1022c, 1022e and 1022g, droplet operations electrodes 1018D and detections spots 1024 form reaction zones 1026, i.e., 1026a through 1026d. Because each reaction zone 1026 includes a detection zone 1024, cross-contamination among droplets in a sequencing protocol is further minimized

Electrode arrangement 1000 may include one or more washing zones 1028 (e.g., washing zones 1028a and 1028b). A permanent magnet (not shown) may be located underneath washing zones 1028a and 1028b. The permanent magnet may be embedded within the deck that holds the droplet actuator when the droplet actuator is mounted on the instrument (not shown). The permanent magnet is positioned in a manner which ensures spatial immobilization of nucleic acid-attached beads during washing between the base additions. Wash buffer fluid may be dispensed from each dedicated wash buffer dispensing electrode 1014 (in the direction of arrows) and collected in each dedicated waste collection electrode 1016 (in the direction of arrows). The arrangement of wash buffer dispensing electrode 1014 and waste collection electrode 1016 improves washing efficiency and reduces time spent in washing. Mixing may be performed in reaction zones 1026 away from the magnet.

The configuration of electrode arrangement 1000 is such that a sample droplet dispensed from sample dispensing reservoir 1010 into circular array 1020 may be transported using droplet operations either clockwise or counterclockwise and combined with a dNTP reaction droplet in reaction zone 1026 (i.e., 1026a or 1026d, respectively). Interspersed dedicated electrode lanes 1022 for wash buffer dispensing (i.e., 1022h) and waste collection (i.e., 1022b and 1022f) may be used to prepare the reaction droplet for subsequent nucleotide incorporation reactions. In one example, a sample droplet may be dispensed from sample dispensing reservoir 1010 into circular array 1020 and transported clockwise using droplet operations into reaction zone 1026a. A dNTP reagent droplet (e.g., dATP reagent droplet) may be dispensed from reagent dispensing electrode 1012a and combined with the sample droplet in reaction zone 1026a to yield a reaction droplet. Incorporation of the nucleotide may be detected as a luminescent signal. After the reaction is complete, the reaction droplet may be transported to washing zone 1028a and washed by addition and removal of wash buffer dispensed from dedicated electrode lane 1022h. This entire sequence constitutes one full pyrosequencing cycle. The reaction droplet may then be transported clockwise to reaction zone 1026b and the sequence of dNTP incorporation, detection and washing repeated using a different dNTP reaction droplet (e.g., dTTP reaction droplet) and adjacent wash buffer dispensing lanes (e.g., dedicated electrode lane 1022d) and washing zone 1028a. The reaction droplet may be transported on circular array 1020 into adjacent reaction zones 1026 and washing zones 1028 any number of times with a user defined sequence of base additions.

In one embodiment, detection of a luminescent signal may be performed by imaging circular array 1020. Because electrode lanes 1022 are dedicated and aligned with a specific detection zone 1024, the position of the luminescent signal in the image is indicative of the dNTP that was incorporated in the pyrosequencing reaction. In another embodiment, individual detection zones 1024 within circular array 1020 may be imaged.

In yet another embodiment, collection electrodes 1016 may be replaced by a single waste collection reservoir within the center of circular array 1020. In this example, washing zones 1026 may be extending into the center of circular array 1020. A masking device, such as a masking tape, may be used to cover the center of circular array 1020 and substantially eliminate any luminescent signal contained in the waste fluid from interfering with the detection of specific signals at detection zones 1024.

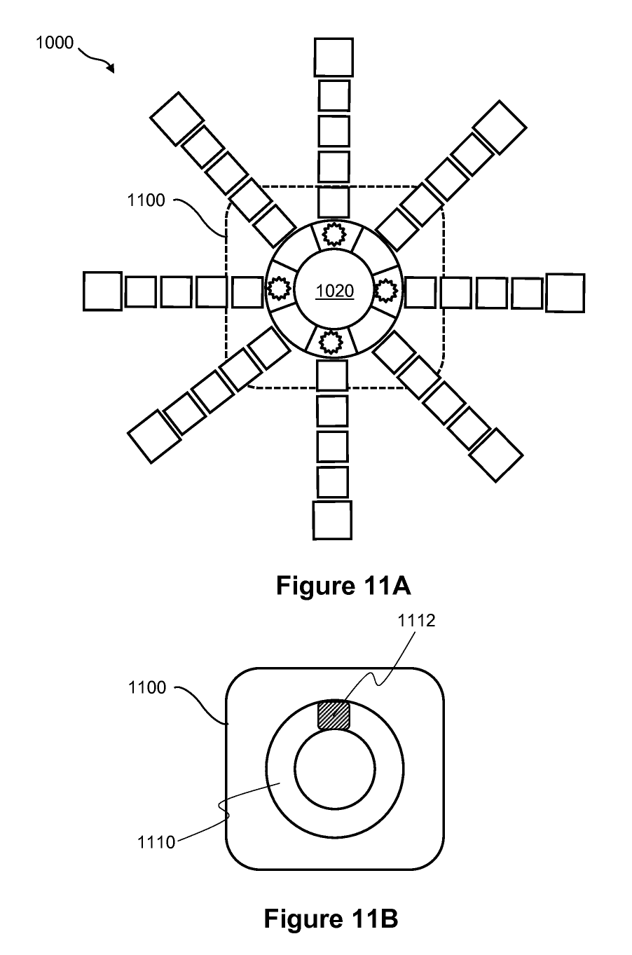

FIG. 11A illustrates a top view of the alignment of the electrode arrangement 1000 of FIG. 10 with a magnetic plate 1100, while FIG. 11B is a top view showing more details of magnetic plate 1100. In this embodiment, a movable magnet is used to transport a sample that includes magnetically responsive beads around a circular array of droplet operations electrodes configured for pyrosequencing. The sample containing the beads may be a unit-sized droplet or may be a much smaller liquid volume hydrating the beads, or may be substantially composed of the beads. For example, the sample may be a single magnetic bead or particle. The sample is transported around a circular array of droplet operations electrodes by magnetic force in the absence of electrowetting forces. The magnetic force may be sufficient to cause the sample to penetrate a meniscus formed between a reagent droplet and the filler fluid. In this case the sample may be rotated in a circular fashion causing it flow through any reagent droplets placed in its circular path. A droplet actuator may be used to insert and remove pyrosequencing reagent droplets (e.g., dNTP droplets that include enzyme mix, APS), wash buffer droplets, and waste droplets in the path of the sample. This may be performed in a synchronized manner so that the sample is rotated through a succession of reagent or wash droplets according to pre-determined user-program. Thus, as the sample rotates through its path it is exposed to a succession of liquids required to perform a DNA sequencing reaction. The sample can be made relatively small compared to the droplets such that a single transit through a wash droplet can result in sufficient washing, and/or the droplet actuator can provide a continual supply of fresh wash droplets (and remove spent was droplets). For example, in one embodiment a circular array of droplets consisting of pyrosequencing reaction droplets for each of the four dNTPs separated by wash droplets is formed. In one transit around the circle the sample would be exposed to each dNTP in turn with washes in between. The timing or location of chemiluminescent signal production could be used to infer the nucleic acid sequence. The droplet microactuator could “reset” the reagent and wash droplets on the circular path for each cycle or after a predetermined number of cycles

Magnetic plate 1100 may, for example, be an acrylic plate. Magnetic plate 1100 may include a circular magnet slot 1110 that may contain a magnet 1112. Magnet 1112 may be a permanent magnet or an electromagnet. Magnet 1112 may be a movable magnet that moves within magnet slot 1110. In one example, the movement of magnet 1112 may be controlled by an actuator that is controlled by a motor.

In operation magnetic plate 1100 may be positioned over electrode arrangement 1000 such that magnet slot 1110 that contains magnet 1112 is aligned with circular array 1020. Magnet 1112 is movable along circular array 1020. In one example, magnet 1112 may be moved in a clockwise direction. As magnet 1112 is moved, beads and a sample (not shown) are transported into and out of reaction and washing zones as described above in reference to FIG. 10. Detection of a luminescent signal may be performed by imaging circular array 1020 or individual detection zones within circular array 1020. In another example, a shadow mask that rotates with magnet 1112 may be used to image luminescent signal only from a sample droplet immobilized within the magnetic field of magnet 1112.

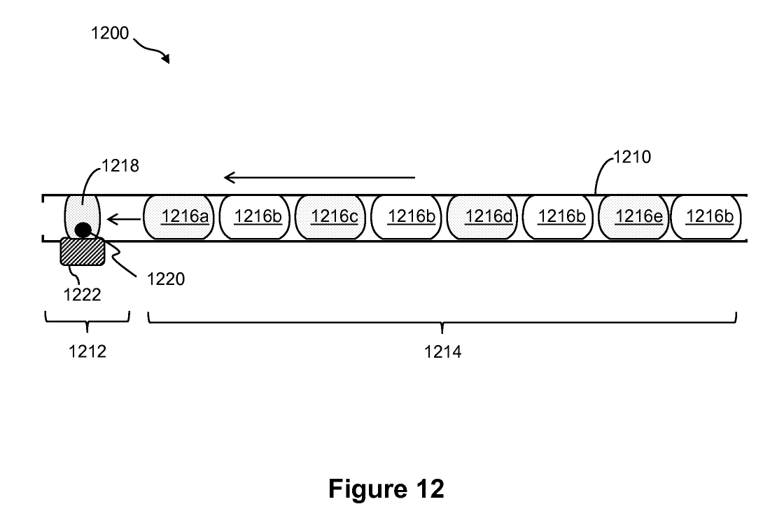

FIG. 12 illustrates a side view of a portion of a capillary device 1200 and an alternative method for performing a pyrosequencing reaction. In this embodiment, a sample droplet that contains magnetically responsive beads may be immobilized in a capillary device. Slugs of reagent and wash fluids may be sequentially moved across the immobilized sample droplet.

Capillary device 1200 may include a capillary tube 1210. Capillary tube 1210 may include a sample loading region 1212 and a fluid loading region 1214. Capillary tube 1210 may be preloaded with one or more slugs of fluid 1216. Slugs of fluid 1216 may, for example, be alternating slugs of reagent fluids and wash buffer fluids. Slugs may be separated with an immiscible fluid, such as an oil, such as a silicon oil. In one example, slugs of fluid 1216 may be an alternating sequence of reagent and wash buffer droplets such as a dATP reagent droplet 1216a, a wash buffer droplet 1216b, a dTTP reagent droplet 1216c, another wash buffer droplet 1216b, a dCTP reagent droplet 1216d, another wash buffer droplet 1216b, a dGTP reagent droplet 1216e, and another wash buffer droplet 1216b. Capillary device 1200 may include fluid paths (not shown) for removing and/or resupplying reagents and/or wash buffer droplets. Alternatively, Capillary device 1200 may have a length which is sufficient to incorporate alternating droplets of all reagents needed to conduct a certain predetermined sequencing protocol.

A sample droplet 1218 may be loaded into capillary tube 1210. Sample droplet 1218 may contain DNA-primer complexes for pyrosequencing. In one example, sample droplet 1218 may contain one or more magnetically responsive beads 1220 that has DNA-primer complexes immobilized thereon. A magnet 1222 may be positioned in proximity of sample droplet 1218 that includes magnetically responsive beads 1220. Magnet 1222 may be a permanent magnet or an electromagnet. Because magnet 1222 is positioned in proximity of sample droplet 1218, magnetically responsive beads 1220 therein are immobilized within the magnetic field of magnet 1222. In another example, magnetically responsive beads 1220 in sample droplet 1218 may be immobilized or otherwise restrained from movement by a physical structure (not shown) within sample loading region 1214. In this example, DNA-primer complexes in sample droplet 1218 may be immobilized on beads that are not magnetically responsive. The droplet slugs may be flowed through the capillary tube across the physically restrained beads.

In operation, sample droplet 1218 with magnetically responsive beads 1220 therein is loaded into sample loading region 1214 of capillary tube 1210 that is preloaded with slugs of fluid 1216. Slugs of fluid 1216 may be sequentially moved over the immobilized beads by application of an external force. In one example, a pressure driven force (e.g., a syringe) may be used to sequentially move slugs of fluid 1216. In another example, vacuum force may be used to sequentially move slugs of fluid 1216. In this example, a vacuum source may be applied to move slugs of fluid 1216. The vacuum source may be released to stop movement of slugs of fluid 1216. Where magnetically responsive beads are used, a magnet may be moved along the capillary tube and/or the capillary tube may be moved relative to the magnet to pull the magnetically responsive beads through the tube, and thus through the reagent and wash slugs in order to execute the protocol. In one example, the magnet (and/or the tube) is moved in zigzag fashion, such that the beads are alternately released from the magnetic field for circulating in the droplet and captured by the magnetically responsive beads for transport through the oil and into the next droplet slug. Droplet slugs may have a length (volume) selected to supply a desired amount of reagent or a desired volume of wash buffer for washing.

8.2 Amplification

The invention provides droplet actuator devices, systems and techniques for amplifying nucleic acids. Thermal cycling is accomplished by cyclically transporting a droplet between fixed temperature zones on the actuator. Thermal cycling is extremely fast because the droplet can be transferred between zones in a fraction of a second while the temperature change within the droplet occurs virtually instantly due its small thermal mass compared to the surrounding system. Examples of droplet actuator configurations, reagents and protocol steps suitable for use with the present invention are described in Pollack et al., U.S. Patent Publication No. 20080038810, entitled “Droplet-Based Nucleic Acid Amplification Device, System, and Method,” published on Feb. 14, 2008, the entire disclosure of which is incorporated herein by reference.

Amplification may be performed extremely rapidly. For example, the inventors have successfully performed 40 cycles of real-time PCR of a Candida albicans target within 5 minutes (7.5 s total cycle time). The inventors have tested the amplification system with a variety of nucleic acid targets including fungi (C. albicans), medically important bacteria (Methicillin-resistant Staphylococcus aureus, Mycoplasma pneumoniae, Echserichia coli), bacterial select agents (Bacillus anthrasis, Franciscella tularensis), and human gene targets (CFTR, RPL4, PCNA). Additionally, multiple formats and variations have been successfully implemented including real-time PCR and reverse transciption PCR (RT-PCR).

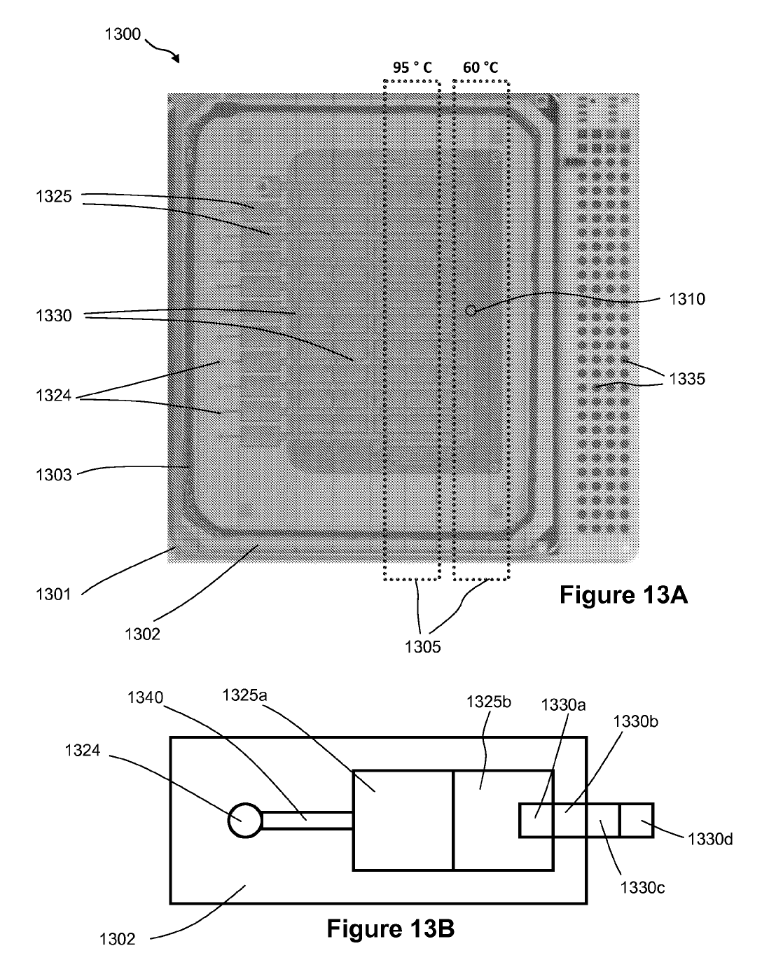

FIGS. 13A and 13B are illustrations of a droplet actuator cartridge 1300. With reference to FIG. 13A, cartridge 1300 includes bottom substrate 1301, which in the illustrated embodiment is made using PCB, but may be made using any suitable material, such as a semiconductive or nonconductive material. Other examples of bottom substrate 1301 materials include glass, silicon and plastic. Cartridge 1300 includes top substrate 1302, which in the illustrated embodiment is made using a glass plate but may be made using any suitable material, such as a semiconductive or nonconductive material. Other examples of top substrate 1302 materials include PCB, silicon and plastic. A preferred top substrate is molded polycarbonate top plate including one or more reservoirs and fluid paths extending from the reservoirs into the droplet operations gap. Reservoirs in the top substrate may, in some embodiments, include a funnel-shaped bottom, terminating in the fluid pathway which opens into the droplet operations gap. The funnel shaped reservoir is useful for reducing dead volume. Bottom substrate 1301 and top substrate 1302 are bound together and sealed by gasket 1303, thereby providing a droplet operations gap between the two substrates. Also shown in FIG. 13A are dispensing reservoir electrodes 1325, droplet transport electrodes 1330, and contact pads 1335, each of which is configured on bottom substrate 1301. Top substrate 1302 also includes a ground on the gap side thereof for grounding or providing a reference potential for droplets in the droplet operations gap. The ground or reference element may be made from any suitable conductor; examples include ITO and PEDOT. PEDOT can be easily applied by spray coating or dip coating or just brushing. The gap-facing surfaces of cartridge 1300 also include a hydrophobic coating, which in the illustrated embodiment is a CYTOP® coating. Contact pads 1335 may be coupled to dispensing reservoir electrodes 1325 and droplet transport electrodes 1330 by wires on the back of the droplet actuator substrate (through vias in the substrate) and are used to electrically couple the droplet actuator to an instrument that controls the electrodes. Substrate 1301 may be manufactured using PCB. One contact pad may be coupled to multiple electrodes, permitting a large number of electrodes to be controlled using only a few contact pads. Openings 1324 in top substrate, which in the illustrated embodiment is made from glass, provide a fluid passage for loading fluid from an exterior of cartridge 1300 into the droplet operations gap in proximity to dispensing reservoir electrodes 1325.

FIG. 13A also illustrates the heater locations 1305 (the heater bars are not shown) and a detection zone 1310. Cartridge 1300 rests on two spring-loaded aluminum heater bars (not shown). Other types of heater mounts may be used. Heaters locations 1305 may be arranged to align with specific areas of the droplet actuator cartridge when it is coupled to the instrument. In the illustrated embodiment, a resistive heater attached to the underside of each bar delivers heat, while a thermistor inserted into the center of the bar is used for closed-loop PID temperature control. When a small offset factor is included to account for a constant temperature difference between the heater bar and the interior of the actuator cartridge 1300, a 300 nL droplet can be temperature controlled to within ±0.5° C. The offset factor may vary depending on chip configuration and may be determined experimentally using a miniature thermocouple inserted into the cartridge and confirmed by thermal simulations. Other types of heater arrangements may be used, for example, see Pollack et al., International Patent Application No. PCT/US2006/047486, entitled “Droplet-Based Biochemistry,” filed on Dec. 11, 2006, the entire disclosure of which is incorporated herein by reference.

FIG. 13B depicts an exploded view of a double electrode reservoir dispensing portion of droplet actuator cartridge 1300, including opening 1324, dispensing reservoir electrodes 1325 (including rear reservoir electrode 1325a and front reservoir electrode 1325b), and droplet transport electrodes 1330 (including transport electrode 1330a, transport electrode 1330b, gate electrode 1330c, and junction electrode 1330d). Channel 1340 provides a fluid passage from an exterior of the droplet actuator into an interior of the droplet operations gap into proximity with dispensing reservoir electrodes 1325. The dimensions of channel 1340 and the on-actuator reservoir atop electrodes 1325a and 1325b are established by gasket 1302. The surfaces of channel 1340 may, in some cases, be hydrophobic—thereby permitting aqueous liquid to be forced into the on-actuator reservoir, and inhibiting the aqueous liquid in the on-actuator reservoir from flowing back out of opening 1324.

One stepwise procedure to dispense a single-sized or double-sized droplet from the double-electrode reservoir may include: (1) front reservoir electrode 1325b ON; (2) transport electrode 1330a (embedded electrode) ON and front reservoir electrode 1325b OFF; (3) transport electrodes 1330a and 1330b ON; (4) transport electrodes 1330a and 1330b, and gate electrode 1330c all ON; (5) transport electrodes 1330a and 1330b, gate electrode 1330c, and junction electrode 1330d all ON (optional, only for double-sized droplet dispensing); and (6) transport electrode 1330b OFF and front reservoir electrode 1325b ON (other electrodes remain ON).

One advantage of having a double electrode reservoir is to allow continuous dispensing of a larger volume of sample with smaller dead volume. By turning the front reservoir electrode 1325b ON and rear reservoir electrode 1325a OFF, the sample (if its footprint is still larger than the area of the front reservoir electrode) always stays in front and overlaps the edges of transport electrode 1330a, which is necessary for reliable dispense. If a traditional single electrode reservoir is used, the sample might drift back after a few dispenses. The sample with reduced volume might fail to touch transport electrode 1330a when the reservoir electrode is ON and further dispensing will be disabled.

Detection for real-time PCR may be performed using a detector, such as a miniature fluorimeter. For example a suitable fluorimeter may include an LED-photodiode pair and filters mounted above the cartridge. The fluorimeter may be configured to illuminate and detect an excitation spot. On substrate 900 in FIG. 9, the detection zone is approximately 500 μm in diameter which is centered within a particular electrode located within the extension temperature zone. For example, in one embodiment, the fluorimeter may be configured to an excitation spot approximately 500 μm in diameter which is centered within a particular 1.125 mm square electrode located within the extension temperature zone. Other types of detector arrangements may be used, for example, see Pollack et al., International Patent Application No. PCT/US2006/047486, entitled “Droplet-Based Biochemistry,” filed on Dec. 11, 2006, the entire disclosure of which is incorporated herein by reference. Any detector orientation (above, below, beside, in the droplet operations gap, etc.) relative to the detection zone may be used, so long as the detector is capable of sensing signal from a droplet at the detection zone.

A cartridge may include multiple droplet transport electrode lanes traversing the two thermal zones. Electrode paths may also provide droplet transport to/from one or more reservoirs for samples, PCR reagents, waste buffers, elution buffers, and waste. An example of a typical droplet operations protocol involves dispensing one 450 nL droplet of sample and one 450 nL droplet of PCR reaction mixture, mixing the droplets together and then thermocycling the combined 900 nL droplet by shuttling it between the two thermal zones according to a user-defined program. The centers of the two zones in the illustrated cartridge are separated by 16 electrodes. Transport rates up to 25 Hz (i.e. electrodes per second) are typically used. Therefore, the droplet was transferred between the two zones in as little as 640 ms.

The invention thus provides a method of thermal cycling a droplet comprising shuttling the droplet between two or more thermal zones wherein the transport time for moving a droplet from one thermal zone to another thermal zone is less than about 5000 ms. In another embodiment, the time that is less than about 4000 ms. In another embodiment, the time that is less than about 3000 ms. In another embodiment, the time that is less than about 2000 ms. In another embodiment, the time that is less than about 1000 ms. In another embodiment, the time that is less than about 500 ms. In one embodiment, the thermal cycling protocol comprises a nucleic acid amplification protocol.

The invention thus provides a method of thermal cycling a droplet comprising shuttling the droplet into or out of a thermal zone wherein the transport time for moving the droplet into or out of a thermal zone is less than about 5000 ms. In another embodiment, the time that is less than about 4000 ms. In another embodiment, the time that is less than about 3000 ms. In another embodiment, the time that is less than about 2000 ms. In another embodiment, the time that is less than about 1000 ms. In another embodiment, the time that is less than about 500 ms. In one embodiment, the thermal cycling protocol comprises a nucleic acid biochemical protocol comprising an incubation step. In one embodiment, the biochemical protocol comprises an affinity assay protocol, such as an immunoassay. In another embodiment, the biochemical protocol includes a thermally mediated reagent activation or deactivation step that comprises transport of the droplet into or out of the thermal zone. The thermal zone may be a heating zone or cooling zone.

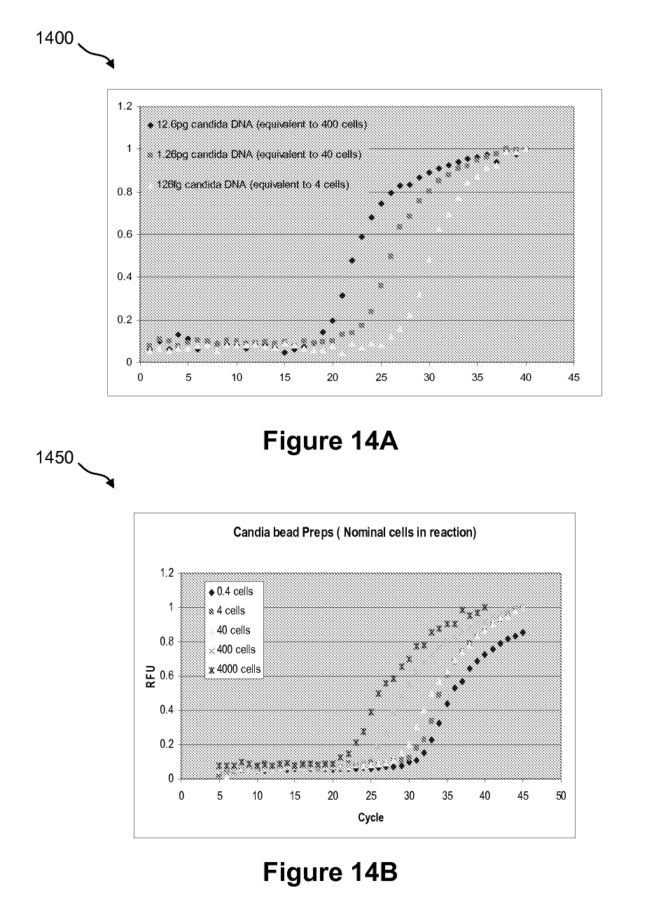

FIGS. 14A and 14B show plots 1400 and 1450, respectively, of real-time PCR curves obtained for a C. albicans model system, indicating that PCR on the cartridge was sensitive and quantitative. The target was a 273-bp fragment of the C. albicans 18S ribosomal RNA gene. The PCR mix consisted of a commercial mix supplemented with extra Taq polymerase and Eva Green dye. The thermal program was 10 s at 94° C. followed by 60 s at 60° C. FIG. 14A shows decade dilutions obtained using genomic DNA. FIG. 14B shows decade dilutions of whole Candida cells spiked into blood and recovered using on off-actuator protocol.

8.3 Software and Systems

Referring to FIGS. 1 through 14, as will be appreciated by one of skill in the art, the invention may be embodied as a method, system, or computer program product. Accordingly, various aspects of the invention may take the form of hardware embodiments, software embodiments (including firmware, resident software, micro-code, etc.), or embodiments combining software and hardware aspects that may all generally be referred to herein as a “circuit,” “module” or “system.” Furthermore, the methods of the invention may take the form of a computer program product on a computer-usable storage medium having computer-usable program code embodied in the medium.

Any suitable computer useable medium may be utilized for software aspects of the invention. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include some or all of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

Computer program code for carrying out operations of the invention may be written in an object oriented programming language such as Java, Smalltalk, C++ or the like. However, the computer program code for carrying out operations of the invention may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network (LAN) or a wide area network (WAN), or the connection may be made to an external computer (for example, through the Internet using an Internet Service Provider).

Certain aspects of invention are described with reference to various methods and method steps. It will be understood that each method step can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the methods.

The computer program instructions may also be stored in a computer-readable memory that can direct a computer or other programmable data processing apparatus to function in a particular manner, such that the instructions stored in the computer-readable memory produce an article of manufacture including instruction means which implement various aspects of the method steps.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing various functions/acts specified in the methods of the invention.

### 9 CONCLUDING REMARKS

The foregoing detailed description of embodiments refers to the accompanying drawings, which illustrate specific embodiments of the invention. Other embodiments having different structures and operations do not depart from the scope of the present invention. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention.

Furthermore, the foregoing description is for the purpose of illustration only, and not for the purpose of limitation.

### 10 REFERENCES

The entire disclosures of the following references are incorporated herein by reference:

Hyman, E., Pyrophosphate-based method and apparatus for sequencing nucleic acids. 1988.

Hyman, E. D., A New Method of Sequencing DNA. Analytical Biochemistry, 1988. 174(2): p. 423-436.

Paik, P., et al., Electrowetting-based droplet mixers for microfluidic systems. Lab Actuator, 2003. 3(1): p. 28-33.

Paik, P., et al., Programmable Flow-Through Real-Time PCR Using Digital Microfluidics. Proc. of MicroTAS, 2007.

Paik, P., V. K. Pamula, and R. B. Fair, Rapid droplet mixers for digital microfluidic systems. Lab on a Actuator, 2003. 3(4): p. 253-259.

Paik, P. Y., et al. Programmable flow-through real-time PCR using digital microfluidics. in MicroTAS. 2007. Paris, France.

Perfect J R, C. A., Fungal Molecular Pathogenesis, in Molecular Principles of Fungal Pathogenesis, H. J, Editor. 2006, ASM Press: Washington, D.C.

Persat F, e.a., Contribution of the (1,3)-Beta-D-glucan assay for diagnosis of invasive fungal infection. J Clin Microbiol 2008. 46: p. 1009-1013.

Pickering J W, e.a., Evaluation of a (1,3)-Beta-D-glucan assay for diagnosis of invasive fungal infections. J Clin Microbiol, 2008. 43: p. 5957-5962.

Pollack, M., Electrowetting-Based Microactuation of Droplets For Digital Microfluidics. 2001, Duke University.

Pollack, M. G., et al. Investigation of electrowetting-based actuation of droplets for integrated microfluidics. in MicroTAS. 2003. Squaw Valley, Calif.

Pounder J I, e.a., Discovering potential pathogens among fungi identified as non-sporulating molds. J. Clin Microbiol, 2007. 45: p. 568-571.

Ronaghi, M., et al., Real-time DNA sequencing using detection of pyrophosphate release. Analytical Biochemistry, 1996. 242(1): p. 84-89.

Ronaghi, M., M. Uhlen, and P. Nyren, A sequencing method based on real-time pyrophosphate. Science, 1998. 281(5375): p. 363-+.

Schloss, J., How to get genomes at one-thousandth the cost. Nature Biotechnology, 2008. 26(10): p. 1113-1115.

Sista, R., et al., Development of a digital microfluidic platform for point of care testing. Lab on a Actuator, 2008. 8: p. 2091-2104.

Sista, R. S., et al., Heterogeneous immunoassays using magnetically responsive beads on a digital microfluidic platform. Lab Actuator, 2008. 8(12): p. 2188-96.

Srinivasan, V., et al. A digital microfluidic biosensor for multianalyte detection. 2003. Kyoto, Japan.

Srinivasan, V., V. K. Pamula, and R. B. Fair, An integrated digital microfluidic lab-on-a-actuator for clinical diagnostics on human physiological fluids. Lab on a Actuator, 2004. 4(4): p. 310-315.

Summerbell R C, e.a., ITS barcodes for Trichophyton tonsurans and T. equinum. Medical Mycology 2007. 45: p. 193-200.

Wolk D M, R. G., Commercial Methods for Identification and Susceptibility Testing of Fungi, in Manual of Commercial Methods in Clinical Microbiology, T. AL, Editor. 2002.

1-25. (canceled)

26. A droplet actuator comprising a PCB substrate comprising electrodes configured for conducting droplet operations, wherein:
(a) the droplet actuator has been subjected to one or more remedial measures effecting reduced background noise caused by PPi contamination relative to a corresponding PCB substrate lacking the remedial measures; and
(b) the remedial measures reduce background noise caused by PPi contamination to an extent sufficient to eliminate undue interference with a pyrosequencing reaction conducted on the droplet actuator using droplets having a volume which is less than about 1 mL.

27. The droplet actuator of claim 26 wherein the droplets having a volume which is less than about 500 μL.

28. The droplet actuator of claim 26 wherein the droplets having a volume which is less than about 50 μL.

29. The droplet actuator of claim 26 wherein the remedial measures may reduce PPi contamination sufficiently to eliminate undue interference of background PPi with detection of PPi generated by a pyrosequencing reaction.

30. The droplet actuator of claim 26 wherein the remedial measures comprise selecting a PCB material manufactured without a pyrophosphate treatment or with a reduced treatment sufficient to eliminate undue interference of background PPi from the PCB with detection of PPi generated by the pyrosequencing reaction.

31. The droplet actuator of claim 26 wherein the remedial measures comprise subjecting the PCB to procedures in the droplet actuator manufacturing process to reduce the introduction of PPi contamination.

32. The droplet actuator of claim 26 wherein the remedial measures comprise washing or otherwise treating the PCB to reduce PPi contamination.

33. The droplet actuator of claim 26 wherein the remedial measures comprise washing or otherwise treating the PCB to reduce PPi contamination using a solution which chemically modifies, inactivates, absorbs and/or removes some or all of the PPi.

34. The droplet actuator of claim 26 wherein the remedial measures comprise washing the PCB in an acid bath to reduce PPi contamination.

35. The droplet actuator of claim 26 wherein the remedial measures comprise treating the PCB with an enzyme to reduce PPi contamination.

36. The droplet actuator of claim 35 wherein the enzyme comprises a pyrophosphatase.

37. The droplet actuator of claim 26 wherein the remedial measures comprise coating the PCB or a region of the PCB with a substance that blocks PPi release.

38. The droplet actuator of claim 37 wherein the substance that blocks PPi release comprises a hydrophobic coating.

39. The droplet actuator of claim 37 wherein:
(a) the substance that blocks PPi release comprises a surface coating selected from the group consisting of: TEFLON® coatings, CYTOP® coatings, silane coatings, and silicone coatings; and
(b) the surface coating having a thickness sufficient to eliminate undue interference of background PPi from the PCB with detection of PPi generated by the pyrosequencing reaction.

40-98. (canceled)

99. A method of detecting PPi release in a droplet on a PCB substrate, the method comprising:
(a) subjecting a PCB substrate to one or more remedial measures effecting reduced background noise caused by PPi contamination of the PCB relative to a corresponding PCB substrate lacking the remedial measures; and
(b) detecting the PPi release in the droplet on the PCB substrate.

100. The method of claim 99 wherein the droplet has a volume which is less than about 500 μL.

101. The method of claim 99 wherein the droplet has a volume which is less than about 50 μL.

102. The method of claim 99 wherein the remedial measures may reduce PPi contamination sufficiently to eliminate undue interference of background PPi with detection of PPi generated by a pyrosequencing reaction.

103. The method of claim 99 wherein the remedial measures comprise selecting a PCB material manufactured without a pyrophosphate treatment or with a reduced treatment sufficient to eliminate undue interference of background PPi from the PCB with detection of PPi generated by the pyrosequencing reaction.

104. The method of claim 99 wherein the remedial measures comprise subjecting the PCB to procedures in the droplet actuator manufacturing process to reduce the introduction of PPi contamination.

105. The method of claim 99 wherein the remedial measures comprise washing or otherwise treating the PCB to reduce PPi contamination.

106. The method of claim 99 wherein the remedial measures comprise washing or otherwise treating the PCB to reduce PPi contamination using a solution which chemically modifies, inactivates, absorbs and/or removes some or all of the PPi.

107. The method of claim 99 wherein the remedial measures comprise washing the PCB in an acid bath to reduce PPi contamination.

108. The method of claim 99 wherein the remedial measures comprise treating the PCB with an enzyme to reduce PPi contamination.

109. The method of claim 108 wherein the enzyme comprises a pyrophosphatase.

110. The method of claim 99 wherein the remedial measures comprise coating the PCB or a region of the PCB with a substance that blocks PPi release.

111. The method of claim 110 wherein the substance that blocks PPi release comprises a hydrophobic coating.

112. The method of claim 110 wherein:
(a) the substance that blocks PPi release comprises a surface coating selected from the group consisting of: TEFLON® coatings, CYTOP® coatings, silane coatings, and silicone coatings; and
(b) the surface coating having a thickness sufficient to eliminate undue interference of background PPi from the PCB with detection of PPi generated by the pyrosequencing reaction.
